(19) United States
(12) Patent Application Publication
(10) Pub. No.: US 2010/0113549 A1
(43) Pub. Date: May 6, 2010

(54) PYRROLIDINE COMPOUNDS

(75) Inventors: Timothy Block, Doylestown, PA (US); Ben Davis, Oxford (GB); Timothy Chapman, Herts (GB); Christopher Schofield, Oxford (GB)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: ISIS INNOVATION LTD., Oxford (GB)

(21) Appl. No.: 11/721,011

(22) PCT Filed: Dec. 6, 2005
(86) PCT No.: PCT/GB2005/004659

§ 371 Date: Jan. 20, 2010

(30) Foreign Application Priority Data
Dec. 6, 2004 (GB) 0426661.5

(51) Int. Cl. A61K 31/40; C07D 207/22; A61P 31/14; A61P 31/12; A61P 3/00; A61P 35/00; A61P 25/00; A61P 19/00; C40B 40/04; C40B 20/00; C07D 405/06; C07D 409/06; C07D 403/06; A61K 31/4025; A61K 31/4178

(52) U.S. Cl. 514/397; 548/537; 514/423; 506/15; 506/2; 548/517; 548/527; 548/314.7; 548/525; 514/422

(57) ABSTRACT

The present invention relates to a process for the preparation of a pyrrolidone compound of the formula (I): where R¹ is substituted or unsubstituted alkyl, alkenyl, or alkynyl, or an aromatic or non-aromatic cyclic or heterocyclic structure, R² is substituted or unsubstituted alkyl or cycloalkyl, and each P is independently a protecting group.

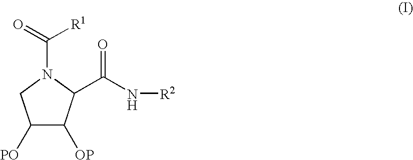

### FIELD OF THE INVENTION

This invention relates to pyrrolidine compounds, and methods for their preparation, which have a variety of uses including as inhibitors of disease-associated targets.

### BACKGROUND TO THE INVENTION

The hydroxylated pyrrolidine scaffold provides sources not only of glyco-mimetics¹ but also of hydroxyproline derivatives². Studies have shown certain bisamide pyrrolidines to be efficient scaffolds for reaction with organometallic reagents³.

Although the Ugi reaction⁴ is widely used in library construction, the use of cyclic imine components in multi-component reactions (MCRs) is rare: in 1989 Joullié demonstrated the role of a single cyanophenoxy dihydropyrryl^{5,6}.

Several important syntheses of dihydroxyproline modules have been reported;⁷ many highlight the difficulty, length, relatively low yields and long reaction times of prolyl amide coupling. Improved access to coupled hydroxyprolines is desirable.

### STATEMENTS OF THE INVENTION

According to the present invention there is provided a process for the preparation of a compound of the formula I:

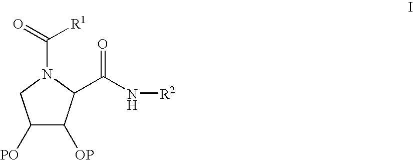

comprising reacting a compound of the formula II:

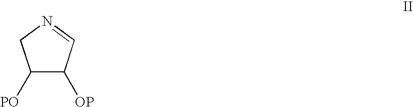

with a compound of the formula:

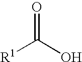

and a compound of the formula:

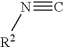

where R¹ is substituted or unsubstituted alkyl, alkenyl, or alkynyl, or an aromatic or non-aromatic cyclic or heterocyclic structure,

R² is substituted or unsubstituted alkyl or cycloalkyl, and each P is independently a protecting group.

Preferred R¹ groups include substituted or unsubstituted C1-C8 alkyl, alkenyl or alkynyl. More preferably, R¹ is substituted or unsubstituted C1-C6 alkyl, alkenyl or alkynyl. Most preferably, R¹ is substituted or unsubstituted C1-C4 alkyl, alkenyl or alkynyl.

An example of a preferred substituted alkyl group is one substituted by a group of the formula R³CONHCHR⁴, where R³ and R⁴ are each independently C1-C4 alkyl, phenyl or benzyl.

Further preferred R¹ groups are substituted or unsubstituted 5- or 6-membered ring structures. The ring may be an alicyclic ring or may include at least one oxygen or nitrogen atom.

Particularly preferred R¹ groups are n-propyl, phenyl or groups of the formula:

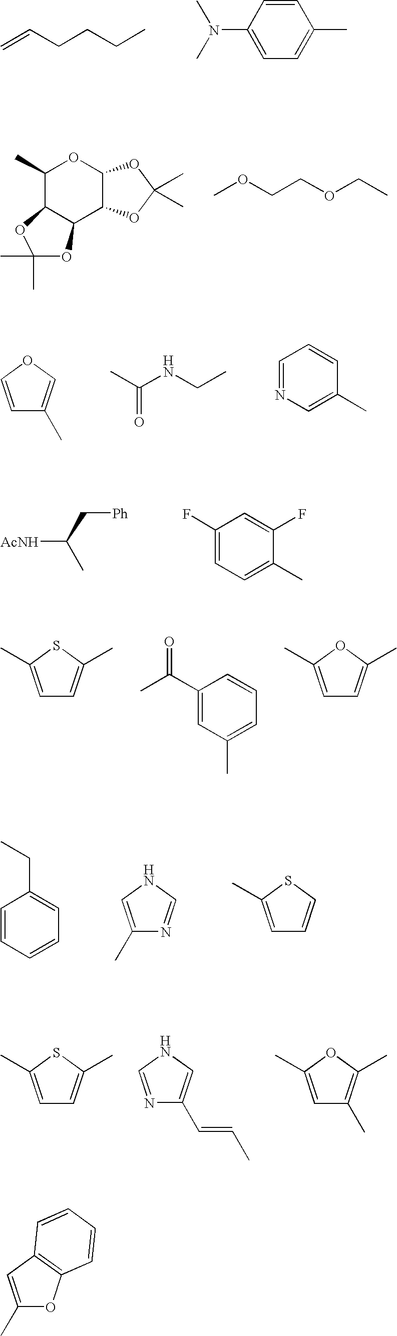

Preferably R² is substituted or unsubstituted C1-C10 alkyl, an example of a substituted alkyl being a C1-C10 alkyl substituted by phenyl.

More preferably R² is i-propyl, n-butyl, t-butyl, n-pentyl, benzyl, cyclohexyl or a group of the formula:

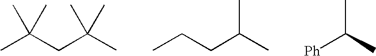

Preferably the group P is i-propyl or teat-butyldimethylsilyl (TBDMS).

Preferably the reaction is carried out in the presence of a non-aqueous solvent, more preferably methanol.

Preferably the compound of the formula I is prepared by dehydrohalogenation of a compound of the formula:

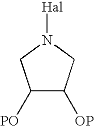

where Hal is halogen. More preferably Hal is chlorine.

Preferably the dehydrohalogenation is carried out in the presence of a ,8-diazabicyclo-[5.4.0]-undec-7-ine (DBU).

Preferably the dehydrohalogenation is carried out in the presence of a non-aqueous solvent, more preferably tetrahydrofuran (THF).

The compound of formula I may be treated to remove the groups P. For instance, the treatment may be with an acid, such as trifluoroacetic acid.

The present invention further provides a compound of the formulas I or II where R¹, R² and P are as defined in Claim 1.

The present invention further provides a compound of formula III:

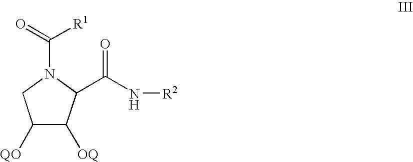

wherein R¹ and R² are as defined herein; and

each Q is independently selected from hydrogen, a salt, protecting group or pharmaceutically acceptable prodrug thereof.

A typical example of a prodrug is an ester form.

Where a protecting group is divalent, both Q groups may be from the same protecting group and thus it is not required that both Q groups are independent of one another, although it is contemplated.

A particularly preferred class of compounds of Formula III have a Formula IIIa below, where both. Q groups are hydrogen:

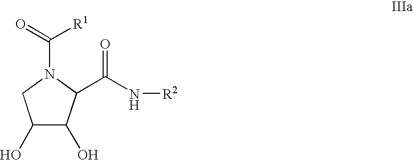

wherein R¹ and R² are as defined herein.

Furthermore, the present invention provides a chemical library comprising two or more different compounds of the formula III. Also provided is a method of identifying a member of the library as an active agent against a particular target, including bringing the library into contact with said target and then determining the effect of each member of the library against a selected property of the target.

The target may be, for instance, a sugar- or peptide-based target, examples being a glycosidase or a glycosyltransferase. The glycosyltransferase may be a glucosylceramide synthase.

Other examples of targets are an HIF hydroxylase, an elastase, hepatitis B virus, hepatitis C virus and bovine diarrhoea virus.

The present invention further provides a compound of the formula III for use as a medicament.

The present invention further provides a pharmaceutical composition comprising a compound of the formula III in combination with a pharmaceutically acceptable carrier, diluent or excipient.

Also provided by the present invention is the use of a compound of the formula III in the manufacture of a medicament for the treatment of a disease with which a target of the compound is associated. The target may be one associated with carbohydrate processing or peptide processing.

The present invention further provides the use of a compound of the formula III in the manufacture of a medicament for the treatment of a lipid storage disease or cancer. Examples of lipid storage diseases include Gaucher's disease and Tay-Sach's disease.

Further provided by the present invention is the use of a compound of the formula III in the manufacture of a medicament for the treatment or prevention of a viral infection. Examples of viral infections which may be treated include viral infections caused by hepatitis B, hepatitis C or bovine diarrhoea virus.

Accordingly, the present invention provides a multi-component reaction (MCR) giving novel bisamide pyrrolidines accessed through a chlorination-elimination strategy³. Subsequent use of the Jouillé-Ugi reaction followed by facile deprotection allows access to wide-ranging aza-sugar/dihydroxy prolyl libraries (Saotome et al., Chem. Biol, 8, 1061 (2001)), which in turn yields potent inhibitors of two disease-associated targets, one based on inhibition of carbohydrate processing and one on peptide processing.

A reaction scheme covering preferred embodiments of the present invention is set out below.

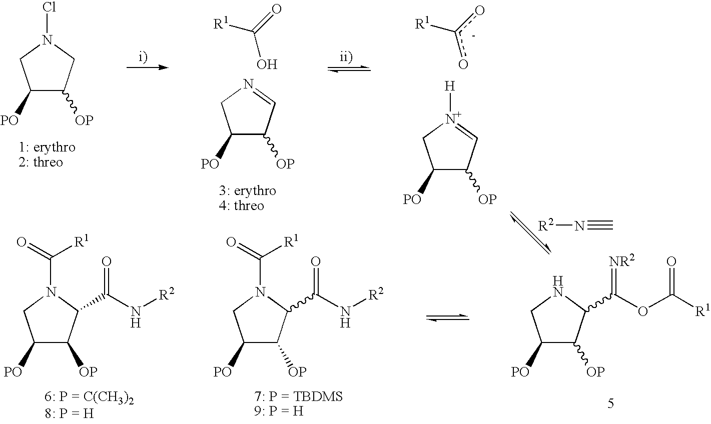

R¹ may be selected from

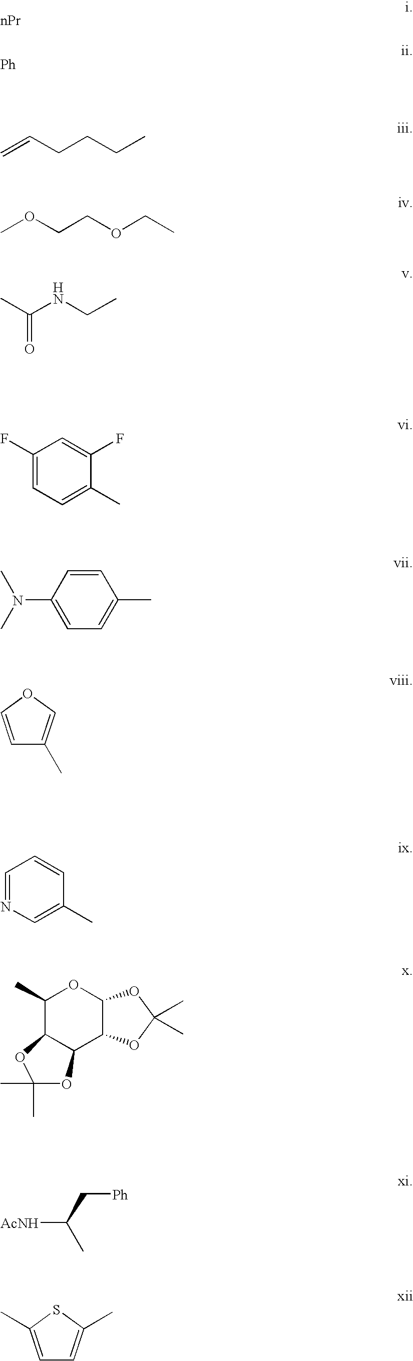

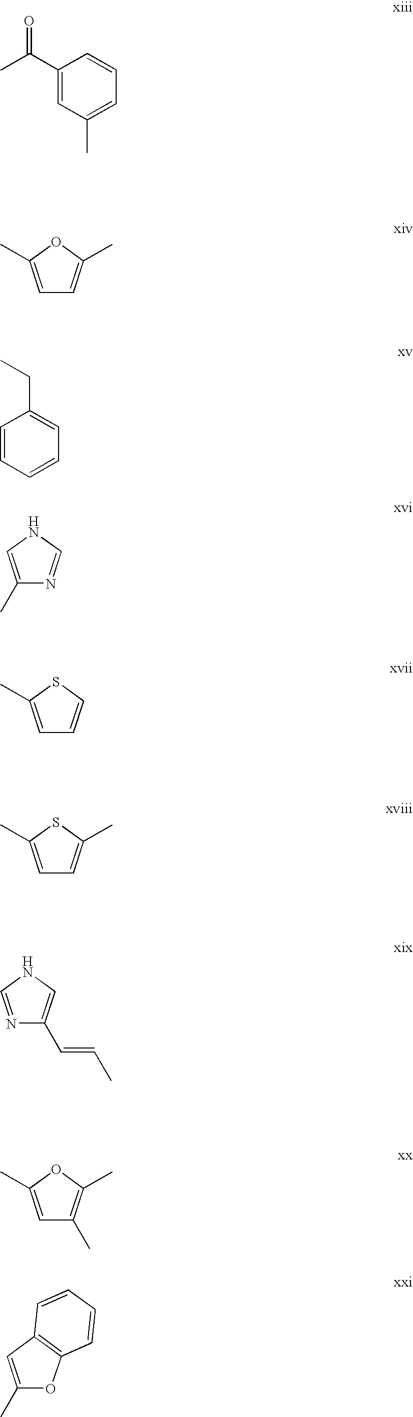

R² may be selected from:

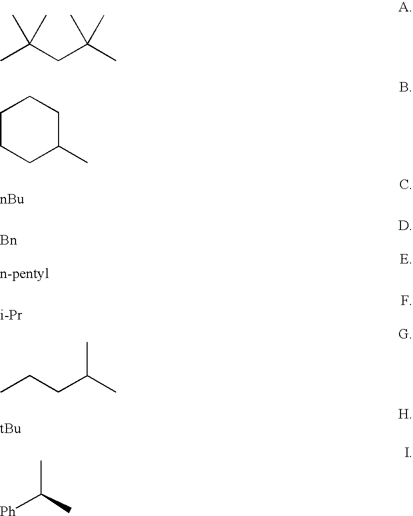

Reaction i) is preferably carried out in the presence of DBU and THF. Reaction ii) is carried out in the presence of carboxylic acid, isocyanide and methanol. Reaction iii) is carried out in the presence of TFA and THF.

Erythritol 3 and threitol 4 imines, formed from treatment of N-chloramine precursors 1 and 2, established the unoptimised viability of reaction with N-acetyl glycine v and benzyl isocyanide D, giving reasonable yields of elaborated bisamide (68 and 64% yield over two steps from 1, 2, respectively). Excellent diastereoselectivity (de >98%) was observed for erythritol 6vD. Deprotection with TFA proceeded smoothly in 90% for erythritol 8vD and 62% for the 2,3-trans threitol species 9vD. Established conditions for ready parallel handling included the removal of isocyanides in vacuo, the removal of acids by base wash and then final treatment with TFA to afford pure deprotected product without recourse to chromatography.

The carboxylic acids i-ix and isocyanides A-H were selected for a library. These included hydrophobic groups which have been shown to enhance the activity of inhibitors of glycosidases, glucosylceramide synthase and prolyl-processing enzymes⁸.

Reaction of 1 with N-acetyl glycine v and isocyanides A-H gave single diastereoisomers in total yields of 43-77%. Compound 1 plus acids i-ix with isocyanide C gave 55-99% yield also as single diastereomers. Similarly, compound 2 gave good to excellent yields (78-98% with v plus A-H and 77-100% with C plus i-ix as a 1:1 mixture of diastereoisomers). Deprotection of all adducts with TFA proceeded quantitatively in most cases. Having established a good level of generality, the library was expanded to 132 deprotected members in total yields of 42-100% from erythritol N-chloramine 1 and 77-100% from threitol N-chloramine 2, all at >90% purity as determined by LC-MS (liquid chromatography-mass spectrometry) and ¹H NMR.

More complex homochiral components were also used, including representative biomolecule fragments. (S)-sec-phenethyl isocyanide I and N-Ac-L-phenylalanine xi gave 51 and 59% yield and >98% de with vii and E, respectively. Protection of sugar hydroxyls is typical in successful MCRs (Sutherlin et at, J. Org. Chem. 61, 8350 (1996); Isenring and Hofheinz, Synthesis, 385 (1981); Hoel and Neilsen, Tetrahedron Letters, 40, 3941 (1999); Liu et al., Bioorg. Med. Chem. Lett. 14, 2221 (2004); Geday et al., Org. Lett, 4, 1967 (2002); 0. Lockhoff, Angew. Chem. Int. Ed. 37, 3436 (1998)) and, indeed, protected D-galacturonic acid x¹⁰ gave 44% overall yield >0.98% de (1+x+E) of azadisaccharide mimic 9xE.

The activities of the array of potential glyco- and peptido-mimetics were probed against 15 different sugar- and peptide-based targets. To test glycomimicry the library was screened against five human glycosidases, five non-mammalian glycosidases and the glycosyltransferase glucosylceramide synthase (GCS), a Gaucher's disease target.¹¹ However, the entire library showed little or no inhibition of glycosidases of 100 μM. This result was unexpected since imino sugars (e.g. NB-DNT) which possess N-linked hydrophobic chains, are potent inhibitors of GCS¹² and pyrrolidine azasugars with hydrophobic ring substituents are effective inhibitors of glycosidases.^{3,8} The importance of a basic endocyclic nitgrogen was tested by treatment of 9iE with 1.5 equivalents of lithium aluminum hydride. This allowed the chemoselective reduction of the tertiary amide in the presence of the secondary amide at C-1 and library elaboration from which 10 and 11 (see formulae below) were successfully identified as GCS inhibitors with IC₅₀ 117 μM and 140 μM respectively.

To test peptide mimicry, the library was screened against peptide-processing targets. Inhibitors of the HIF hydroxylases are of current interest with respect to developing anti-ischemic agents¹³ and elastases are implicated in several diseases such as pancreatitis, rheumatoid arthritis and emphysema. These enzymes preferentially accept peptides/protein substrates that contain proline residues: factor inhibiting hypoxia-inducible factor¹⁴ (FIH, which catalyses hydroxylation of VNAP motifs), PHD2 (Iaakola et al., Science, 292, 468 (2001); Ivan et al., 292, 464 (2001); Epstein et al., Cell, 107, 43 (2001); Bruick and Knight, 294, 1337 (2001); Schofield and Ratcliffe Nat. Rev. Mol. Cell Biol. 5, 343 (2004)) (one isoform of the prolyl hydroxylase domain containing hydroxylases) and porcine pancreatic elastase (PPE).

The library was tested in whole pathogen assays against hepatitis B virus (HBV) and bovine diarrhoea virus (BVDV), which is a primary model of human HCV.15 A specific pattern of potency against BVDV for aromatic R¹ and branched R² substituents (as in 9iiA, 9viiG and 9viiiF) emerged. IC₅₀ of 25 μM (9iiA) and 30 μM (9viiG, 9viiiF, MOI=0.5) compare very favourably with NN-DNJ, 10 μM, MOI=0.1 and better than those for NB-DNJ (125 μM, MOI=0.1).¹⁶ The formulae for 9iiA, 9viiG and 9viiiF are given below.

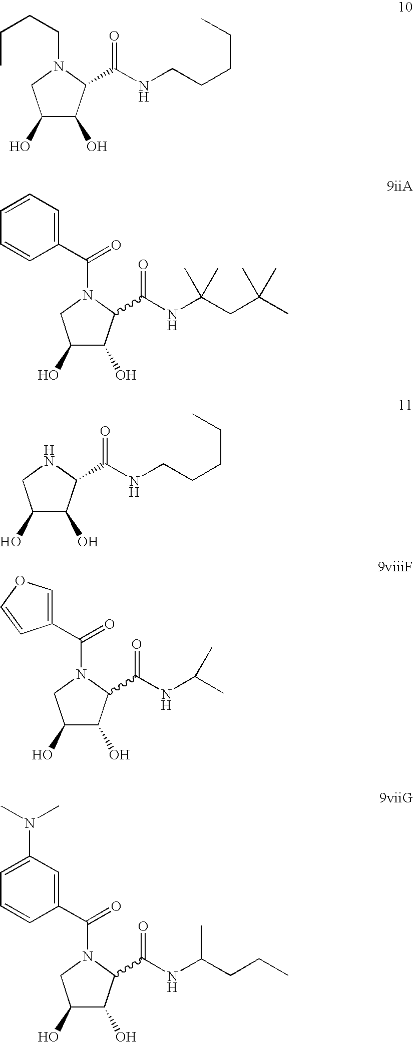

Reduction of viral protein E2 level, lack of glycosidase and HBV inhibition also indicated a novel, selective mechanism distinct from those of these previous imino sugars¹⁶. This is believed to be the first example of a BVDV inhibiting azasugar that does not affect HBV. No significant toxicity was observed even at highest concentration (300 μM).

Other exemplary compounds of the present invention, which may be optionally synthesised as part of a library are shown below:

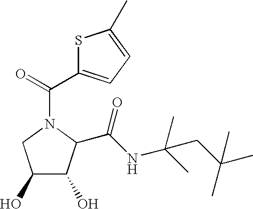

¹H NMR 0.8-1.1, 1.22-1.45 (17H, chain), 2.5 (3H, s, ArMe), 3.39-3.75 (2H, m, Ha, Hb), 4.05-4.77 (3H,m, Hc, Hd, He), 6.8 (1H, m, Ar), 7.6 (1H, m, Ar)

LCMS ESI (+ve) RT=18.2 expected 383 [M+H]+, found 383 [M+H]+ (10%), 405 [M+Na]+ (100%).

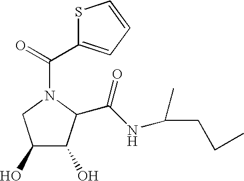

¹H NMR 0.9-1.7 (10H, m, chain), 3.50-3..85 (2H, m, Ha, Hb)3.89 (1H, m, NCH), 4.10-4.85 (3H, m, He, Hd, He), 7.15 (1H, m, Ar), 7.7-7.9 (2H, m, Ar)

LCMS ESI (+ve) RT=14.0 expected 327 [M+H]+, found 327 [M+H]+ (100%).

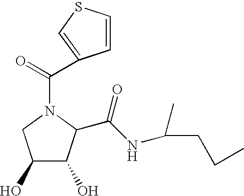

¹H NMR 0.9-1.7 (10H, m, chain), 3.23-3.85 (2H, m, Ha, Hb) 3.85-4 (1H,m, CHN), 4.15-4.80 (3H, m, Hc, Hd, He), 7.5 (2H,m Ar), 8.2 (1H, m, Ar)

LCMS ESI (+ve) RT=13.5 expected 327 [M+H]+, found 327 [M+H]+ (80%).

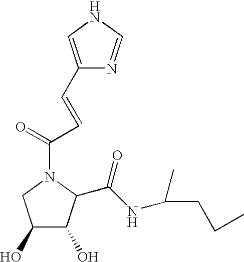

¹H NMR 0.7-1.7 (10H, m, chain), 3.12-3.82 (2H, m, Ha, Hb) 3.85 (1H, m, NCH), 4.05-4.75 (3H, m, Ho, Hd, He), 6.6, 7.0, 7.2, 7.5 (2H, 4×m, 2×HC═) 7.95 (1H, m, Ar), 9.05 (1H, m, Ar)

LCMS ESI (+ve) RT=5.0 expected 337 [M+H]+, found 337 [M+H]+ (90%).

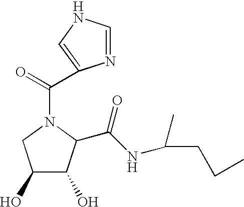

¹H NMR 0.8-1.8 (10H, m, chain), 3.2-3.80 (2H, m, Ha, Hb), 3.85 (1H, m, NCH), 4.15-4.60 (3H, m, Hc, Hd, He), 8.2 (1H, m, Ar), 9.1 (1H, m, Ar)

LCMS ESI (+ve) RT=5.6 expected 311 [M+H]+, found 311 [M+H]+ (100%)

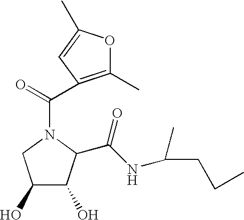

¹H NMR 0.9-1.6 (10H, m, chain), 2.12-2.34 (3H, m. Me), 2.36, 2.5 (3H, m, Me), 3.20-3.73 (2H, m, Ha, Hb) 3.85-4.02 (1H, m, NCH), 4.03-4.68 (3H, m, Hc, Hd, He) 6.2 (1H, m, Ar)

LCMS ESI (+ve) RT=15.3, 15.8 expected 339 [M+H]+, found 339 [M+H]+ (10%), 361 [M+Na]+ 100%).

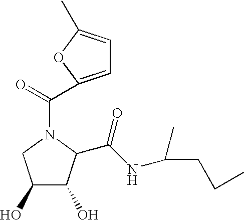

¹H NMR 0.85-1.90 (10H, m, chain), 3.10-3.85 (2H, m, Ha, Hb) 3.87-4.05 (1H, m, NCH), 4.05-4.55 (3H, m, Hc, Hd, He) 6.6 (1H, m, Ar), 7.20 (1H, m, Ar).

LCMS ESI (+ve) RT=14.5, 15.0 expected 389 [M+H]+, found 411 [M+Na]+ (100%), 389 [M+H]+ (40%).

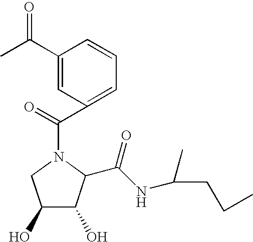

¹H NMR 0.9-1.6 (10H, m, chain), 2.46-2.78 (3H, m. Me), 3.20-3.75 (2H, m, Ha, Hb) 4.00 (1H, m, NCH), 4.05-4.48 (3H, m, Hc, Hd, He) 7.4-8.85 (4H, m, Ar)

LCMS ESI (+ve) RT=15.3, 15.8 expected 339 [M+H]+, found 339 [M+H]+ (10%), 361

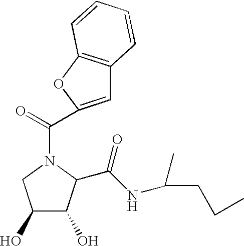

¹H NMR 0.6-1.8 (10H, m, chain), 3.42-3.89 (2H, m Ha, Hb) 4.0 (1H, m, NCH), 7.3-7.9 (5H, m, Ar)

LCMS ESI (+ve) RT=15.6, 16.0 expected 361 [M+H]+, found 361 [M+H]+ (10%), 383 [M+Na]+ (100%).

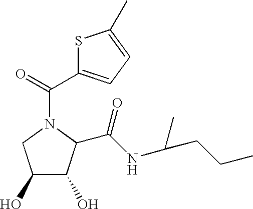

¹H NMR. 0.7-1.8 (10H, m, chain), 2.5 (3H, s, 1×Me), 3.32-3.75 (2H, m, Ha, Hb) 4.95 (1H, m, NCH), 4.08-4.55 (3H, m, He, Hd, He), 6.85 (1H, m, Ar), 7.7 (1H, m, Ar)

LCMS ESI (+ve) RT=14.2, 14.9 expected 341 [M+H]+, found 341 [M+H]+ (5%), 363 [M+Na]+ (100%).

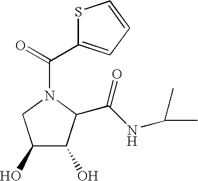

¹H NMR 1-1.5 (6H, m, chain), 3.40-3.90 (2H, m, Ha, Hb), 4.05-4.10 (1H, m, NCH), 4.10-4.50 (3H, in, Hc, Hd, He), 7.15 (1H, m, Ar), 7.7-7.9 (2H, m, Ar)

LCMS ESI (+ve) RT=9.2, 10.0 expected 299 [M+H]+, found 299 [M+H]+ (5%), 321[M−H+Na]+ (100%).

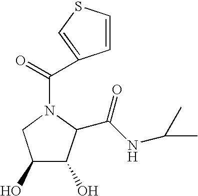

¹H NMR 0.9-1.5 (6H, m,chain), 3.24-3.74 (2H, m, Ha, Hb), 4.00 (1H, m, NCH), 4.10-4.50 (3H, m, Hc, Hd, He) 7.45 (2H, m, Ar), 8.25 (1H, m, Ar)

LCMS ESI (+ve) RT=9.7, 10.1 expected 299 [M+H]+, found 299 [M+H]+ (50%).

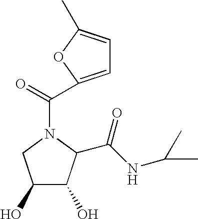

¹H NMR 1.0-1.4 (6H, m, chain), 3.30-3.75 (2H, m, Ha, Hb), 4.0 (1H, m, NCH), 4.05-4.82 (3H, m, Ho, Hd, He), 6.6 (1H, m, Ar), 7.2 (1H, m, Ar)

LCMS ESI (+ve) RT=11.8 expected 363 [M+H]+, found 363 [M+H]+ (20%), 385 [M−H+Na]+ (100%).

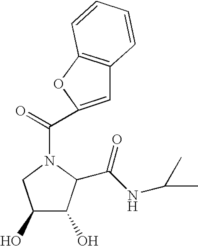

¹H NMR 0.8-1.5 (6H, m, chain), 3.23-3.85 (2H, m, Ha, Hb), 4.05 (1H, m, NCH), 4.10-4.45 (3H, m, Hc, Hd, He) 7.2-7.9 (5H, m, Ar)

LCMS ESI (+ve) RT=12.8, 13.6 expected 333 [M+H]+, found 333 [H+H]+ (5%), 355 [M−H+Na]+ (100%).

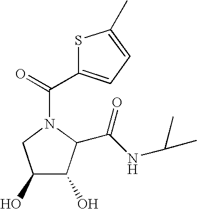

¹H NMR 0.8-1.4 (6H, m, chain), 2.4 (3H, s, Me), 3.2-3.78 (2H, m, Ha, Hb) 3.8-4.0 (1H, m, NCH), 4.0-4.75 (3H, m, Hc, Hd, He) 6.8 (1H, m, Ar), 7.6 (1H, m, Ar)

LCMS ESI (+ve) RT=11.3, 12.2, expected 313 [M+H]+, found 313 [M+H]+ (5%), 335 [M−H+Na]+ (100%).

### REFERENCES

¹a) B. Winchester, G. W. J. Fleet, Glycobiology 1992, 2, 199; b) B. Winchester, S. al Daher, N. C. Carpenter, I. Cenci di Bello, S. S. Choi, A. J. Fairbanks, G. W. J. Fleet, Biochem. J. 1993, 290, 743.

²a) A. Lazaro, L. Garcia, C. R. D. Correia, Tetrahedron Lett. 2003, 44, 1553; b) T. K. Chakraborty, P. Srinivasu, S. K. Kumar, A. C. Kunwar, J. Org. Chem. 2002, 67, 2093.

³a) T. M. Chapman. S. Courtney, P. Hay, B. G. Davis, Chem. Eur, J. 2003, 9, 3397; b) B. G. Davis, M. A. T Maughan, T. M. Chapman, R. Villard, S. Courtney, Org. Lett. 2002, 4, 103.

⁴a) I. Ugi, C. Steibrückner, Chem. Ber. 1961, 94, 734; b) I. Ugi, Angew. Chem. Int. Ed. 1962, 1, 8.

⁵M. M. Bowers, P. Carroll, M. M. Jouillé, J. Chem. Soc. Perkin Tran s.l 1989, 857.

⁶More recently tetrahydropyridines have been used vs aldehyde/amine condensate; W. Maison, A Liitzen, M. Kosten, I. Schlemminger, O. Westerhoff, J. Martens, J. Chem. Soc. Perkin Trans. 1 1999, 3515.

⁷a) C. M. Taylor, C. A. Weir, J. Org. Chem. 2000, 65, 1414; b) C. L. Jenkins, L. E. Bretscher, I. A. Guzei, R. T. Raines; J. Am. Chem. Soc. 2003, 125, 6422; c) G. Kalaus, N. Malkieh, M. Kajtár-Peredy, J. Brlik, L. Szabó, C. Szántay, J. Org. Chem. 1988, 53, 42,

⁸a) R. H. Furneaux, G. Limberg, P. C. Tyler, V. L. Schramm, Tetrahedron 1997, 53, 2915; b) G. B. Evans, R. H. Furneaux, T. L. Hutchison, H. S. Kesar, P. E. Morris, V. L. Schramm, P. C. Tyler, J. Org. Chem. 2001, 66, 5723; c) A. Lohse, K. B. Jensen, K. Lundgren, M. Bols, Bioorg. Med. Chem. 1999, 7, 1965; d) P. A. Wright, R. C. Wilmouth, I. J. Clifton, C. J. Schofield, Eur. J. Biochem 2001, 268, 2969.

⁹a) T. D. Butters, L. A. G. M. van den Brock, G. W. J. Fleet, T. M. Krulle, M. R. Wormald, R. A. Dwek, F. M. Platt, Tetrahedreon: Assymmetry 2000, 11, 113; b) J. H. Fernandez, M. A. F. Hayashi, A. C. M. Camrgo. G. Neshich, Biochem. Biophys. Res. Commun. 2003, 308, 219.

¹⁰H. Y. Godage, A. J. Fairbanks, Tetrahedron. Lett. 2000, 41, 7589.

¹¹A. R. Sawkar, W-C. Cheng, E. Beutler, C-H. Wong, W. E. Balch, J. W. Kelly, Proc. Natl. Acad. Sci. 2002, 99, 15428.

¹²F. M. Platt, G. R. Neises, G. B. Karlsson, R. A. Dwek, T. D. Butters, J. Biol. Chem. 1994, 269, 27108.

¹³K. Hewitson, L. McNeill, C. J. Schofield, Curr. Pharm. Des. 2004, 10, 821.

¹⁴a) K. S. Hewitson, L. A. McNeill, M. V. Riordan, Y. M. Tian, A. N. Bullock, R. W. Welford, J. M. Elkins, N. J. Oldham, S. Battacharya, J. M. Gleadle, P. J. Ratcliffe, C. W. :Pugh, C. J. Schofield, J. Biol. Chem. 2002, 277, 26351 b) D. Lando, D. J. Peet, D. Whelan, J. Gorman, M. Whitelaw, Science 2002, 295, 858.

¹⁵N. Zitzmann, A. S. Mehta, S. Carrouee, T. D. Butters, F. M. Platt, J. McCauley, B. Blumberg, R. A. Dwek, T. M. Block, Proc. Natl. Acad. Sci. 1999, 96, 11878.

¹⁶D. Durantel, N. Branza-Nichita, S. Carrouée-Durantel, T. D. Butters, R. A. Dwek, N. Zitzmann, J. Virol. 2001, 75, 8987.

The disclosure includes the described diols in all their forms, including for example their isomers, prodrugs and pharmaceutically acceptable salts, as well as the hydrates, solvates and co-crystals of the diols and of their isomers, salts and prodrugs.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The extent of protection includes counterfeit or fraudulent products which contain or purport to contain a compound of the invention irrespective of whether they do in fact contain such a compound and irrespective of whether any such compound is contained in a therapeutically effective amount. Included in the scope of protection therefore are packages which include a description or instructions which indicate that the package contains a pharmaceutical formulation of the invention and a product which is, or purports to be, such a formulation.

Substituents

A substituent is halogen or a moiety having from 1 to 30 plural valent atoms selected from C, N, O, S and Si as well as monovalent atoms selected from H and halo. In one class of compounds, the substituent, if present, is for example selected from halogen and moieties having 1, 2, 3, 4 or 5 plural valent atoms as well as monovalent atoms selected from hydrogen and halogen. The plural valent atoms may be, for example, selected from C, N, O, S and B, e.g. C, N, S and O.

Included is a class of compounds in which the substituent is selected from cyano, azo, unsubstituted or substituted hydrocarbyl containing or not containing one or more in-chain —O— linkages, and unsubstituted or substituted heterocyclyl, halogen, -GR⁷, —SGR⁷, —OGR⁷, -Q-C(R^{5a}R^{5b})—NR¹R², —NO₂, -G¹NR³R⁴, —NR¹⁰GR⁷, —SO₂R⁷, —SO₂NR³R⁴,
wherein:
Q is NR^{f} or a direct bond, where R^{f} is selected from H, hydroxy, unsubstituted or substituted hydrocarbyl (e.g. C₁-C₄ alkyl), unsubstituted or substituted hydrocarbyloxy, and NR^{17a}R^{17b}, where R^{17a} and R^{17b} are each independently selected from H, hydroxy, unsubstituted or substituted hydrocarbyl (e.g. C₁-C₄ alkyl), unsubstituted or substituted hydrocarbyloxy (e.g. C₁-C₄ alkoxy); and R¹⁶ is H or unsubstituted or substituted hydrocarbyl (e.g. C₁-C₄ alkyl);
R¹ and R² are each independently selected from H, hydroxy, unsubstituted or substituted hydrocarbyl (e.g. C₁-C₄ alkyl), unsubstituted or substituted hydrocarbyloxy, and NR^{8a}R^{8b}, where R^{8a} and R^{8b} are each independently selected from H, hydroxy, unsubstituted or substituted hydrocarbyl (e.g. C₁-C₄ unsubstituted or substituted hydrocarbyloxy (e.g. C₁-C₄ alkoxy); and R⁹ is H or unsubstituted or substituted hydrocarbyl (e.g. C₁-C₄ alkyl);
R³ and R⁴ are each independently selected from hydrogen, OH, unsubstituted or substituted hydrocarbyl, or unsubstituted or substituted hydrocarbyloxy;
R^{5a} and R^{5b} together form ═N—R¹⁰, ═O, ═S or R^{5a} and R^{5b} are the same or different and selected from hydrogen, OR¹¹, —NR¹⁶R¹⁷, —S—R¹¹, unsubstituted or substituted hydrocarbyl, (for example alkyl e.g. lower alkyl, alkenyl e.g. lower alkenyl, aryl or cycloalkyl) or unsubstituted or substituted hydrocarbyloxy (for example alkoxy e.g. lower alkoxy, or aryloxy), carboxy, halo,
   wherein:
   R¹⁰ is selected from hydrogen, OH, unsubstituted or substituted hydrocarbyl, or unsubstituted or substituted hydrocarbyloxy,
   R¹¹ is selected from hydrogen and unsubstituted or substituted hydrocarbyl; and
   R¹⁶ and 17 are each independently selected from hydrogen, OH, unsubstituted or substituted hydrocarbyl, or unsubstituted or substituted hydrocarbyloxy (for example, R¹⁰, R¹⁶ and R¹⁷ are each independently selected from H, hydroxy, C₁-C₄ alkyl, C₁-C₄ alkoxy);
R⁷ is selected from hydrogen, and unsubstituted or substituted hydrocarbyl, except that -GR⁷ may not be hydrogen,

G is C═O, —C(0)O— or a direct bond;

G¹ is C═O or a direct bond.

Further to be mentioned is a class of compounds in which substituents include, but are not limited to, amino, mono- or di-lower alkyl substituted amino, wherein the lower alkyl substituents may be unsubstituted or further substituted by those substituents listed above for alkyl groups, N-lower alkanoylamino, N,N-di-lower alkanoylamino, halogen (especially fluoro, chloro, bromo or iodo), lower alkyl, halo-lower alkyl (especially trifluoromethyl), hydroxy, esterified carboxy, etherified hydroxy, lower alkoxy, lower alkanoyl, lower alkanoyloxy, halo-lower alkoxy (especially 2,2,2-trifluoroethoxy), amino-lower alkoxy (especially 2-amino-ethoxy), nitro, cyano, mercapto, lower alkylthio, carboxy, lower alkoxycarbonyl, carbamoyl, amidino, guanidino, ureido, halo-lower alkylthio, heterocyclyl, heteroaryl, heterocyclylalkyl, heteroarylalkyl, lower alkanoyl, carbamoyl, and N-mono- or N,N-di-lower alkyl substituted carbamoyl, N-(hydroxy-lower alkyl)-carbamoyl (especially N-(2-hydroxyethyl)-carbamoyl), wherein the lower alkyl substituents may be unsubstituted or further substituted

Exemplary substituent moieties are example halogen, hydroxy, alkyl, alkenyl, alkynyl, cycloalkyl, haloalkyl (e.g. trifluoromethyl), alkoxy, carboxy, amino or NO₂, each moiety being unsubstituted or substituted (where chemically I0 possible).

Included are compounds in which a substituent contains one or a combination of moieties selected from categories 1), 2) and 3) below and optionally one or more moieties selected from category 4) below:
1) aliphatic moieties, in particular having from 1 to 7 carbon atoms, e.g. 1, 2, 3 or 4, particularly alkyl or alkenyl moieties, e.g. alkyl;
2) carbocyclic rings, which may be saturated or unsaturated (e.g. aromatic), particularly to be mentioned are bicyclic and monocycle rings and especially monocycle rings having 5 or 6 ring members;
3) heterocyclic rings, which may be saturated or unsaturated (e.g. aromatic), particularly to be mentioned are bicyclic and monocycle rings and especially monocycle rings having 5 or 6 ring members;
4) linking moieties selected from O, N, Si and C(O), wherein two or more linking moieties may be combined to form a larger linking group for example C(O)O, C(O)NH or OC(O)NH.

In these compounds, a plurality of moieties selected from 1), 2) and 3) may be linked together either directly or through a linking moiety 4). Of course, one compound may contain one or more linking moieties. Tri- or more valent linking moieties such as N and Si may serve to link together just two moieties selected from 1), 2) and 3), in which case the remaining valencies are suitably occupied by hydrogen; alternatively N or Si may link together three said moieties, or Si may link together four said moieties. Where a substituent contains a plurality of moieties selected from 1), 2) and 3), the moieties may be the same of different and may independently be selected from categories 1), 2) and 3).

Moieties 1), 2) and 3) may be unsubstituted or substituted by one or more substituents selected from, in particular, hydroxy, amino, amidino, guanidino, hydroxyguanidino, formamidino, isothioureido, ureido, mercapto, C(O)H or other lower acyl, lower acyloxy, carboxy, sulfo, sulfamoyl, carbamoyl, cyano, azo, or nitro, which hydroxy, amino, amidino, guanidino, hydroxyguanidino, formamidino, isothioureido, ureido, mercapto, carboxy, sulfo, sulfamoyl, carbamoyl and cyano groups are in turn unsubstituted or substituted on at least one heteroatom by one or, where possible, more C₁-C₇ aliphatic groups. For example, the substituent may have 0, 1, 2, 3, or 4 such substituents; sometimes there are a larger number of substituents as can happen, for example, for one or more perfluorinated alkyl or cyclic groups, e.g. CF₃, as well as other optional substituents.

Particular moieties 1), 2) and 3) to mention are straight chain and branched alkyl, 5- and 6-membered carbocyclic rings (notably phenyl and cyclohexyl), and 5- and 6-membered heterocyclic rings (notably 5-membered rings containing a single heteroatom, e.g. furan, thiophene, pyrrole; and 6-membered rings containing one or two heteroatoms, e.g. piperidine, piperazine, morpholine, pyridine, pyrimidine and pyrazine).

Definitions

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

The term “halogen” herein includes reference to F, Cl, Br and I. In some instances halogen is Cl. In one class of compounds, halogen is F.

Hydrocarbyl may be defined as having for example up to 20 carbon atoms, especially up to 12 carbon atoms. Hydrocarbyl groups may be linear or branched aliphatic, e.g. alkyl, alkenyl or alkynyl; they may be alicyclic (i.e. aliphatic-cyclic), e.g. cycloalkyl; they may be aromatic, e.g. phenyl. Hydrocarbyl groups may contain a combination of two or more moieties selected from aliphatic, alicyclic and aromatic moieties, e.g a combination of at least one alkyl group and an aromatic group.

Alkyl may have up to 20, for example up to 12 carbon atoms and is linear or branched one or more times; preferred is lower alkyl, especially preferred is C₁-C₄-alkyl, in particular methyl, ethyl or i-propyl or t-butyl, where alkyl may be substituted by one or more substituents. Unsubstituted alkyl, preferably lower alkyl, is especially preferred.

The term “alkenyl” as used herein refers to a straight or branched chain alkyl moiety having from two to six carbon atoms and having, in addition, at least one double bond, of either E or Z stereochemistry where applicable. This term refers to groups such as ethenyl, 2-propenyl, 1-butenyl, 2-butenyl, 3-butenyl, 1-pentenyl, 2-pentenyl, 3-pentenyl, 1-hexenyl, 2-hexenyl and 3-hexenyl and the like.

The term “alkoxy” as used herein refers to an unsubstituted or substituted straight or branched chain alkoxy group containing from one to six carbon atoms. This term refers to groups such as methoxy, ethoxy, propoxy, isopropoxy, butoxy, tert-butoxy, pentoxy, hexoxy and the like.

The term “alkynyl” as used herein refers to a straight or branched chain alkyl moiety having from two to six carbon atoms and having, in addition, at least one triple bond. This term refers to groups such as ethynyl, 1-propynyl, 2-propynyl, 1-butynyl, 2-butynyl, 3-butynyl, 1-pentynyl, 2-pentynyl, 3-pentynyl, 1-hexynyl, 2-hexynyl and 3-hexynyl and the like.

The term “substituted” as used herein in reference to a moiety or group means that one or more hydrogen atoms in the respective moiety, especially up to 5, more especially 1, 2 or 3 of the hydrogen atoms are replaced independently of each other by the corresponding number of the described substituents. The substituents may be the same or different and may be selected from hydroxy, halogen (e.g. fluorine), hydroxyalkyl (e.g. 2-hydroxyethyl), haloallcyl (e.g. trifluoromethyl or 2,2,2-trifluoroethyl), amino, substituted amino (e.g. N-alkyllamino, N,N-dialkylamino or

N-alkanoylamino), alkoxycarbonyl, phenylalkoxycarbonyl, amidino, guanidino, hydroxyguanidino, formamidino, isothioureido, ureido, mercapto, acyl, acyloxy, carboxy, sulfo, sulfamoyl, carbamoyl, cyano, azo, nitro and the like.

The term “substituted” as used herein in reference to a moiety or group means that one or more hydrogen atoms in the respective moiety, especially up to 5, more especially 1, 2 or 3 of the hydrogen atoms are replaced independently of each other by the corresponding number of the described substituents.

It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled man.

Substituted alkyl may therefore be, for example, alkyl as last defined, may be substituted with one or more of substituents, the substituents being the same or different and selected from hydroxy, etherified hydroxyl, halogen (e.g. fluorine), hydroxyalkyl (e.g. 2-hydroxyethyl), haloalkyl (e.g. trifluoromethyl or 2,2,2-trifluoroethyl), amino, substituted amino (e.g. N-alkyllamino, N,N-dialkylamino or N-alkanoylamino), alkoxycarbonyl, phenylalkoxycarbonyl, amidino, guanidino, hydroxyguanidino, formamidino, isothioureido, ureido, mercapto, acyl, acyloxy such as esterified carboxy for example, carboxy, sulfo, sulfamoyl, carbamoyl, cyano, azo, nitro and the like.

The term “lower” when referring to substituents such as alkyl, alkoxy, alkyl amine, alkylthio and the like denotes a radical having up to and including a maximum of 7, i.e. C₁, C₂, C₃, C₄, C₅, C₆ or C₇ especially from 1 up to and including a maximum of 4, carbon atoms, the radicals in question being unbranched or branched one or more times.

Cycloalkyl is a cyclic group of 3 or more in-ring carbon atoms, for example C₃, C₄, C₅, C₆ or C₇. Cyclo alkyl may be substituted. Cycloalkyl may be, in particular, a linking group between two moieties.

Halo-lower alkyl, halo-lower alkyloxy, halo-lower alkylthio and the like refer to substituents having an alkyl portion wherein the alkyl portion is mono- to completely substituted by halogen. Halo-lower alkyl, halo-lower alkyloxy, halo-lower alkylthio and the like are included within substituted lower alkyl, substituted lower alkoxy, substituted lower alkylthio and the like.

An amino group is a nitrogen containing moiety, usually with at least two of its substitution sites occupied by hydrogen. An amino group having less than two substitution sites occupied by hydrogen is refereed to as a mono- or di-substituted amino moiety. This may be defined where the amino is substituted or substituted by a hydrocarbyl moiety, the hydrocarbyl moiety being, for example, selected from lower alkyl, especially C₁, C₂, C₃ or C₄ alkyl and thus may be mono- or di-lower alkyl amino, cycloalkyl, especially cyclohexyl, alkyl-carboxy, carboxy, lower alkanoyl, especially acetyl, a carbocyclic group, for example cyclohexyl or phenyl, a heterocyclic group; where the hydrocarbyl moiety is unsubstituted or substituted by, for example lower alkyl (C₁, C₂, C₃, C₄, C₅ , C₆ or C₇), halogen, OH, esterified carboxy, etherified hydroxy, lower alkoxy, NH₂, SH, S-alkyl, SO-alkyl, SO₂-alkyl, NH-alkyl, N-dialkyl, carboxyl, CF₃, wherein alkyl may be unsubstituted or substituted branched, unbranched or cyclic C₁₋₆, interrupted 0-3 times by O, S, N.

The alkyl portion of lower alkyl, lower alkoxy, mono- or di-lower alkyl amino, lower alkyl thio and other substituents with an alkyl portion is especially C₁-C₄alkyl, for example n-butyl, sec-butyl, tert-butyl, n-propyl, isopropyl, methyl or ethyl. Such alkyl substituents are unsubstituted or substituted by halogen, hydroxy, nitro, cyano, lower alkoxy, C₃, C₄, C₅, C₆ or C₇ cycloalkyl, amino, or mono- or di-lower alkyl amino, unless otherwise indicated.

As used herein, the term mercapto defines moieties of the general structure —S—Rₑ wherein Rₑ is selected from H, alkyl, a carbocylic group and a heterocyclic group as described herein.

As used herein, the term guanidino defines moieties of the general structure —NHR—C(NH)NH₂ and derivatives thereof, in particular, where hydrogen is replaced by alkyl, e.g. methyl or ethyl.

As used herein, the term amidino defines moieties of the general structure —C(NH)NH₂ and derivatives thereof, in particular, where hydrogen is replaced by alkyl, e.g. methyl or ethyl.

A cyclic group is either a carbocyclic group or a heterocyclic group. Both carbocyclic and hererocyclic moieties may be aromatic or non aromatic. The cyclic group can be mono- bi- or tri-cyclic. A monocyclic group comprises one ring in isolation. A bicyclic group is a fused-ring moiety joined either at a common bond or at a common atom, thus providing a spino moiety. A bicyclic group may comprise two aromatic moieties, one aromatic and one non-aromatic moiety or two non-aromatic moieties. Cycloalkyl is a cyclic group.

A heterocyclic moiety is for example an aromatic ring or ring system having 16 or fewer members, preferably a ring of 5 to 7 members. Heterocycle also includes a three to ten membered non-aromatic ring or ring system and preferably a five- or six-membered non-aromatic ring, which may be fully or partially saturated. In each case the rings may have 1, 2 or 3 hetero atoms selected from the group consisting of nitrogen, oxygen and sulfur. The heterocycle is unsubstituted or substituted by one or more, especially from one to three, for example one, identical or different substituents. Important substituents on heterocycle are those selected from the group consisting of halogen, for example, fluorine or chlorine; mono- or di-lower alkyl-substituted amino wherein the alkyl groups are unsubstituted or substituted by halogen, hydroxy, nitro, cyano, lower alkoxy, C₃-C₇ cycloalkyl, a heterocyclic radical or a heteroaryl radical; lower alkyl, such as methyl or ethyl; halo-lower alkyl, such as trifluoromethyl; lower alkoxy, such as methoxy or ethoxy; halo-lower alkoxy, for example, trffluoromethoxy; lower alkylthio, such as methylmercapto, halo-lower alkylthio, such as trifluoromethylthio, a heteroaryl radical, heteroaryl-lower-alkylene, a heterocyclic radical or heterocyclic-lower-alkylene.

Heterocyclylalkyl is as cycloalkyl but containing one or more in-ring heteroatoms and may be exemplified by piperidyl, piperazinyl, pyrollidine, morpholinyl.

Etherified hydroxy is, for example, alkoxy, especially lower alkoxy. The lower alkyl moiety of lower alkoxy is unsubstituted or substituted by one or more, preferably one, radicals such as e.g. amino, N-lower alkylamino, N,N-di-lower alkylamino, N-lower alkanoylamino, N,N-di-lower alkanoylamino, hydroxy, lower alkoxy, lower alkoxy-lower alkoxy, lower alkanoyl, lower alkanoyloxy, cyano, nitro, carboxy, lower alkoxycarbonyl, carbamoyl, amidino, guanidino, ureido, mercapto, lower alkylthio, halogen or a heterocyclic radical.

As used herein, the term “product” or “product of the invention” relates to any product containing a compound of the present invention. In particular the term product relates to compositions containing a compound of the present invention, such as a pharmaceutical composition, for example.

The term “pharmaceutical composition” as used herein may be taken to mean a composition that may be administered to a mammalian host by means of oral, parenteral, topical, or rectal administration, or by inhalation. A pharmaceutical composition as disclosed herein may contain additives such as carriers (both active and passive), diluents, adjuvants and the like.

The term “parenteral” as described herein includes administration by injection such as subcutaneous injection, intravenous injection, intramuscular injection, intracisternal injection and other infusion procedures.

The term “therapeutically effective amount” as used herein may be determined by definition that it relates to an amount of a drug, or pharmaceutical agent that will provide the desired therapeutic response of a mammal (animal or human).

Hydrocarbyl and hydrocarbyloxy groups may be substituted, for example by one or more substituents selected from lower alkyl, halogen, OH, esterified carboxy, etherified hydroxy, lower alkoxy, lower alkyl thio, NH₂, mono- or di-substituted amino, carboxy, CF₃, SH, S-alkyl, SO-alkyl, SO₂-alkyl, wherein alkyl may be unsubstituted or substituted branched, unbranched or cyclic C₁₋₆, interrupted 0-3 times by O, S, N.

An example of a substituted hydrocarbyl group is haloalkyl, e.g. C₁-C₄ haloalkyl.

An example of a substituted hydrocarbyloxy group is haloalkoxy, e.g. C_{I}-C₄ haloalkoxy.

Hydrocarbyl is preferably lower alkyl, lower alkenyl or lower alkynyl. Hydrocarbyloxy is preferably lower alkoxy.

Lower alkyl is, for example, methyl, ethyl, n-propyl, isopropyl, n-butyl, isobutyl, sec-butyl, tert-butyl, n-pentyl, isopentyl, neopentyl, n-hexyl or n-heptyl.

Lower alkylene is, for example, methylene (—CH₂—), ethylene (—CH₂—CH₂—), propylene (—CH₂—CH₂—CH₂—) or tetramethylene (—CH₂—CH₂—CH₂—CH₂—).

Halogen is especially fluorine, chlorine, bromine or iodine, more especially fluorine, chlorine or bromine, in particular fluorine.

Cycloalkyl is preferably C₃-C₁₀-cycloalkyl, especially cyclopropyl, dimethylcyclopropyl, cyclobutyl, cyclopentyl, cyclohexyl or cycloheptyl, cycloalkyl being unsubstituted or substituted by one or more, especially 1 to 3, substituents.

Esterified carboxy is especially lower alkoxycarbonyl, such as tert-butoxycarbonyl, iso-propoxycarbonyl, methoxycarbonyl or ethoxycarbonyl, phenyl-lower alkoxycarbonyl, or phenyloxycarbonyl.

Alkanoyl is primarily alkylcarbonyl, especially lower alkanoyl, e.g. acetyl. In particular, the alkanoyl group may be substituted by substituents, e.g. CO—R.

A cyclic group can be substituted or unsubstituted. Appropriate substituents include, but are not limited to, amino, mono- or di-lower alkyl substituted amino, wherein the lower alkyl substituents may be unsubstituted or further substituted by those substituents listed above for alkyl groups, N-lower alkanoylamino, N,N-di-lower alkanoylamino, halogen (especially fluoro, chloro, bromo or iodo), lower alkyl, halo-lower alkyl (especially trifluoromethyl), hydroxy, esterified carboxy, etherified hydroxy, lower alkoxy, lower alkanoyl, lower alkanoyloxy, halo-lower alkoxy (especially 2,2,2-trifluoroethoxy), amino-lower alkoxy (especially 2-amino-ethoxy), nitro, cyano, mercapto, lower alkylthio, carboxy, lower alkoxycarbonyl, carbamoyl, amidino, guanidino, ureido, halo-lower alkylthio, heterocyclyl, heteroaryl, heterocyclylalkyl, heteroarylalkyl, lower alkanoyl, carbamoyl, and N-mono- or N,N-di-lower alkyl substituted carbamoyl, N-(hydroxy-lower alkyl)-carbamoyl (especially N-(2-hydroxyethyl)-carbamoyl), wherein the lower alkyl substituents may be unsubstituted or further substituted.

A carbocyclic moiety which is alicyclic especially comprises 3, 4, 5, 6 or 7 in ring carbon atoms and is non aromatic, but may be saturated or unsaturated. Preferred alicyclic groups comprise cycloalkyl groups, which are preferably C₃-C₁₀-cycloalkyl, especially cyclopropyl, dimethylcyclopropyl, cyclobutyl, cyclopentyl, cyclohexyl or cycloheptyl, cycloalkyl being unsubstituted or substituted by one or more, especially 1, 2 or 3, substituents.

An aromatic carbocyclic group preferably has a ring system of not more than 16 carbon atoms and is preferably mono- bi- or tri-cyclic and may be fully or partially substituted, for example substituted by at least two substituents. Preferably, the aromatic group is selected from phenyl, naphthyl, indenyl, azulenyl and anthryl.

A substituted aromatic group is generally an aromatic group that is substituted with from 1-5, preferably 1 or 2, substituents.

A heterocyclic moiety especially is a radical selected from the group consisting of oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyol, thiazolyl, isothiazolyl, dithiazolyl, oxazolyl, isoxazolyl, pyridyl, pyrazinyl, pyrimidinyl, piperidyl, especially piperidin-1-yl, piperazinyl, especially piperazin-1-yl, pyridazinyl, morpholinyl, especially morpholino, thiomorpholinyl, especially thiomorpholino, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, each of these radicals being unsubstituted or substituted by one to two radicals selected from the group consisting of lower alkyl, especially methyl or tert-butyl, lower alkoxy, especially methoxy, and halo, especially bromo or chloro.

Salts and Prodrugs

The compounds of the disclosure may be administered in the form of pharmaceutically acceptable salts. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The disclosure may therefore also include pharmaceutically-acceptable salts of the disclosed compounds as hereinbefore described and as hereinafter described, for example, wherein the parent compound is modified by making acid or base salts thereof. For example the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The invention includes prodrugs of the aforementioned compounds, which can be metabolically converted to the subject compounds by the recipient host. As used herein, a prodrug is a compound that exhibits pharmacological activity after undergoing a chemical transformation in the body. An example of such a prodrug is a pharmaceutically acceptable ester of a carboxylic acid.

The invention includes prodrugs for the active pharmaceutical species of the invention, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents compounds which are rapidly transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996); and The organic chemistry of drug design and drug action by Richard B Silverman in particular pages 497 to 546; each of which is incorporated herein by reference.

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. acyloxyalkyl esters, amides |
| Alcohol | Esters, including e.g. sulfates and phosphates as |
| | well as carboxylic acid esters |
| Amine | Amides, carbamates, imines, enamines, |
| Boronic acid | Diol ester |
| Carbonyl (aldehyde, | Imines, oximes, acetals/ketals, enol esters, |
| ketone) | oxazolidines and thiazoxolidines |

Prodrugs also include compounds convertible to the active drug by an oxidative or reductive reaction. As examples may be mentioned:
Oxidative activation
   N- and O-dealkylation
   Oxidative deamination
   N-oxidation
   Epoxidation
Reductive activation
   Azo reduction
   Sulfoxide reduction
   Disulfide reduction
   Bioreductive alkylation
   Nitro reduction.

Also to be mentioned as metabolic activations of prodrugs are nucleotide activation, phosphorylation activation and decarboxylation activation.

The prodrugs may be used, for example, to increase solubility, stability, permeability, or to control efflux.

Other prodrugs may be carrier-linked or modified to enhance usability of active transport mechanisms. In particular, the prodrugs are pharmaceutically acceptable salts, esters or solvates.

### DETAILED DESCRIPTION OF THE INVENTION

The invention will now be further described by way of examples only. The following abbreviations will be used:

APO Atmospheric pressure chemical ionisation

DBU 1,8-Diazabicyclo-[5.4.0]undec-7-ene

d.r. Diastereomeric ratio

ES Electrospray ionisation

EtOAc Ethyl acetate

eq Molar equivalent(s)

HRMS High resolution mass spectrometry

IR Infra red

LC-MS Liquid chromatography-mass spectrometry

NMR Nuclear magnetic resonance

nOe Nuclear Overhauser effect

SM Starting material

THF Tetrahydrofuran

TFA Trifluoroacetic acid

TLC Thin Layer Chromatography

General Experimental

¹H and ¹³C nuclear magnetic resonance (NMR) spectra were recorded on Varian Gemini 200, Unity 300, VXR 400, Varian Inova 500 or Bruker AMX 500 NMR spectrometers at the frequencies indicated. Where indicated, NMR peak assignments were made using COSY, DEPT, or NOESY experiments; all others are subjective. All chemical shifts are quoted on the δ-scale and were referenced to residual solvent as an internal standard. Combinations of the following abbreviations are used to describe NMR spectra: s, singlet; d, doublet; t, triplet; q, quartet; m, multiplet; br, broad, obs; obscured; r; roofing; p, pseudo. Infra-red spectra were recorded on a Perkin-Elmer Fourier Transform spectrophotometer. The following abbreviations are used to describe infra red absortion bands: br, broad; s, strong. Mass spectra were recorded using electron impact, chemical ionisation or electrospray ionisation techniques on Micromass Autospec or LCT mass spectrometers; high resolution electrospray spectra were recorded by the UK EPSRC mass spectrometry service at Swansea, UK. Thin layer chromatography (TLC) was carried out on aluminum sheets coated with silica gel 60F₂₅₄ (Merck, 1.05554). Plates were developed using an ethanolic phosphomolybdic acid or aqueous basic potassium permanganate dip. Flash column chromatography was performed using silica gel (Merck, 60A, 230-400 Mesh). Tetrahydrofuran was distilled immediately prior to use under N₂ over Na. Methanol was distilled from magnesium/iodine, and stored over 4 Å sieves under nitrogen. All solvents were removed by evaporation under reduced pressure.

Imine Formation: General Procedure:

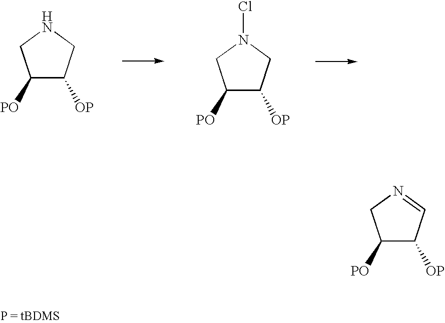

1.1 eq N-chlorosuccinamide (0.96 g) was dissolved in anhydrous diethyl ether (50 ml) and the reaction mixture was stirred together at room temperature until tic (30% EtOAc in petrol and 10% MeOH in EtOAc) implied complete reaction (ca. 2-4.5 hours). The reaction mixture was then filtered, washed with water (50 ml), dried over MgSO₄ and concentrated by evaporation until ca. 10 ml of the solution remained. This solution was then used immediately for the formation of the imine without further analysis.

Imine Formation—NaH Method

The ˜10 ml of chloramine solution from the above reaction was taken, and 1.1 eq NaH (60% dispersion in mineral oil, 0.130 g) was carefully added portion-wise (H₂ gas evolved). The reaction mixture was stirred until no further gas was evolved. The reaction mixture was then spun in a centrifuge and the imine-containing solution was decanted and concentrated carefully under vacuum. The residual imine was then dissolved in methanol (23.5 ml) and used crude, and without further analysis, for the next step. The approximate concentration of the imine solution is assumed to be 50 ug (1 eq) per 1.3 ml.

Imine Formation—DBU Method

The ˜10 ml of chloramine solution from the above reaction was taken, and 3 eq DBU (1.3 ml) was carefully added. The reaction mixture was stirred until tlc (30 EtOAc in petrol) implied complete consumption of starting material (ca 2-4 hours). The reaction mixture was then filtered and the imine-containing solution was concentrated carefully under vacuum. The residual imine was then dissolved in methanol (23.5 ml) and used crude, and without further analysis for the next step. The approximate concentration of the imine solution is assumed to be 50 ug (1 eq) per 1.3 ml.

Library Synthesis—General Procedure.

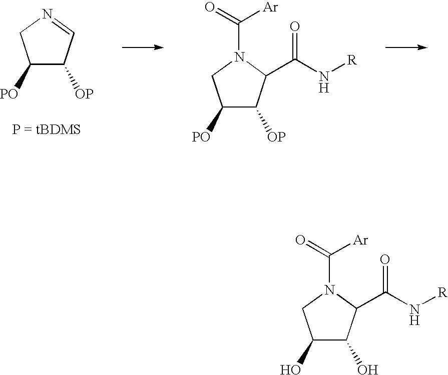

18×3 eq. acid were weighed out into reaction vials. 1.3 ml stock imine solution was added to each reaction vial, followed by 3 eq of isocyanide. The reaction mixtures were then agitated for ca. 70 hours and concentrated by evaporation in a genvac system. The obtained residues were dissolved in ethyl acetate (10 ml), washed with sodium bicarbonate solution (7 ml), dried over magnesium sulphate, filtered and concentrated by evaporation. The residues were deprotected by dissolving the samples in 2 ml methanol, adding 100 ul HCl and agitating for a further 40 hours. The samples were then concentrated by evaporation and analysed by ¹H NMR and LCMS.

### 1-(N-Acetyl-glycinyl)-2,3-trans-2-benzylcarbamoyl-3,4-cis-O-isopropylidene-pyrrolidine (6vD)

DBU (0.066 mL, 67 mg, 0.44 mmol, 1.3 eq) was added to a solution of N-chloro-2,3-O-isopropylidene-1,4-dideoxy-1,4-iminoerythritol 1 (60 mg, 0.34 mmol, 1.0 eq) in dry THF (3 mL) under Ar. The reaction mixture was stirred for 4 h, then filtered under Ar to remove DBU.HCl. The filtrate was concentrated under reduced pressure to yield an oil. This was dissolved in dry MeOH (2 mL) under Ar and N-acetyl glycine (51 mg, 0.44 mmol, 1.3 eq) and benzyl isocyanide (0.053 mL, 51 mg, 0.44 mmol, 1.3 eq) were added. After 40 h stirring TLC (EtOAc) revealed consumption of imine and formation of major product (R_{f} 0.2). The reaction mixture was concentrated to dryness; EtOAc (30 mL) was added and the mixture was washed with NH₄Cl (aq.) (10 mL), then NaHCO₃ (aq.) followed by brine (10 mL). The organic layer was dried (Na₂SO₄) and concentrated under reduced pressure to yield 6vD as a pale yellow oil (87 mg, 68%). NMR experiments showed the product to be a single diastereomer with minor rotameric effects causing 2 signals in ¹H NMR. nOe showed the product to have trans stereochemistry. ¹H NMR (400 MHz, CDCl₃, COSY, nOe): δ=1.30, 1.40 (2×s, 2×3H; 2×C(CH₃)), 1.98 (s, 3H; CH₃CO), 3.71 (m, 2H; NCH₂), 3.97 (dd, ²J(H,H)=17.5 Hz, ³J(H,H)=4.3 Hz, 1H; CH₃CONHCHH′), 4.05 (dd, ²J(H,H)=17.5 Hz, ³J(H,H)=4.3 Hz, 1H; CH₃CONHCHH′), 4.32 (dd, ²J(H,H)=15.0 Hz, ³J(H,H)=5.6 Hz, 1H; NHCHH′Ph), 4.44 (dd, ²J(H,H)=15.0 Hz, ³J(H,H)=6.1 Hz, 1H; NHCHH′Ph), 4.75 (s, 1H; NCH), 4.88 (m, 1H; NCH₂CH), 4.99 (d, ³J(H,H)=6.0 Hz, 1H; NCHCH), 6.53 (br s, 1H; NHCH₂), 7.11 (pt, ³J(H,H)=5.6 Hz, 1H; NHCH₂), 7.22-7.42 (m, 5H; C₆H₅); ¹³C NMR (100 MHz, CDCl₃, HMQC): δ=22.8 (CH₃CO), 24.7, 26.8 (2×C(CH₃)), 41.9 (CH₃CONCH₂), 43.4 (NCH₂Ph), 52.2 (NCH₂), 66.2 (NCH), 79.4 (NCH₂CH), 80.9 (NCHCH), 112.1 (C(CH₃)₂)), 127.6, 128.7, 129.0 (3×aromatic CH), 137.7 (quaternary aromatic C), 168.3, 168.5, 170.3 (3×amide C═O); IR (film): ν̅=3076 (aromatic C—H), 2990, 2936 (aliphatic C—H), 1651 cm⁻¹ (3×amide C═O); MS (ES⁺): m/z (%): 398 (100) [M+Na]⁺, 376 (36) [M+H]⁺; HRMS (ES⁺): m/z: calcd for C₁₉H₂₆N₃O₅: 376.1872; found: 376.1889 [M+H]⁺.

### 1-(N-Acetyl-glycinyl)-2-(benzylcarbamoyl)-3,4-trans-O-di-tert-butyldimethylsilyl-pyrrolidine (7vD)

DBU (0.086 mL, 87 mg, 0.57 mmol, 3.0 eq) was added to a solution of N-chloro-2,3-O-tert-butyldimethylsilyl-1,4-dideoxy-1,4-iminothreitol 2 (70 mg, 0.19 mmol, 1.0 eq) in dry THF (3 mL) under Ar. The reaction mixture was stirred for 4 h then the mixture was filtered under Ar to remove DBU.HCl. The filtrate was concentrated under reduced pressure to yield an oil. This was dissolved in dry MeOH (2 mL) under Ar and N-acetyl glycine (67 mg, 0.57 mmol, 3.0 eq) and benzyl isocyanide (0.070 mL, 67 mg, 0.574 mmol, 3.0 eq) were added. After 40 h stirring TLC (EtOAc/petrol 4:1) revealed consumption, of imine and formation of major product. The reaction mixture was concentrated to dryness, EtOAc (30 mL) was added and the mixture was washed with NH₄Cl (aq.) (10 mL), then NaHCO₃ (aq.) followed by brine (10 mL). The organic layer was dried (Na₂SO₄) and concentrated under reduced pressure to yield a pale yellow oil (97 mg). This crude product was purified by flash column chromatography on silica gel (EtOAc/petrol 4:1), which allowed separation of diastereomers to afford the 2,3-trans diastereomer 7vDa (34 mg, 32%) and 2,3-cis diastereomer 7vDb (34 mg, 32%) (total mass=68 mg, 64%). Each diastereomer was analysed by nOe NMR experiments and this allowed determination of which product corresponded to which stereochemistry. 7vDa: ¹H NMR (500 MHz, CDCl₃, COSY, nOe): δ=0.07, 0.07, 0.15, 0.16 (4×s, 4×3H; 4×SiCH₃), 0.86, 0.88 (2×s, 2×9H; 2×SiC(CH₃)₃), 1.99 (s, 3H; CH₃CO), 3.41 (d, ²J(H,H)=10.1 Hz, 1H; NCHH′CHOSi), 3.72 (dd, ²J(H,H)=10.1 Hz, ³J(H,H)=3.8 Hz, 1H; NCHH′CHOSi), 3.89 (dd, ²J(H,H)=17.8 Hz, ³J(H,H)=3.9 Hz, 1H; CH₃CONHCHH′), 4.09 (d, ³J(H,H)=3.8 Hz, 1H; NCH₂CHOSi), 4.11 (m, 2H; NHCHH′Ph, CH₃CONHCHH′), 4.43 (s, 1H; NCHCHOSi), 4.55 (s, 1H; NCHCHOSi), 4.70 (dd, ²J(H,H)=15.0 Hz, ³J(H,H)=7.4 Hz, 1H; NHCHH′Ph), 6.47 (br s, 1H; 1 of NHCH₂), 6.58 (pt, ³J(H,H)=5.0 Hz, 1H; 1 of NHCH₂), 7.24-7.36 (m, 5H; C₆H₅); ¹³C NMR (100 MHz, CDCl₃, HMQC): δ=−5.1, −4.9, −4.8 (Si(CH₃)2), 17.9, 18.0 (SiC(CH₃)₂), 22.9 (CH₃CO), 25.6, 25.7 (SiC(CH₃)₃), 42.3 (CH₃CONCH₂), 43.4 (NCH₂Ph), 53.7 (NCH₂CHOSi), 69.4 (NCHCHOSi), 76.3 (NCH₂CHOSi), 81.3 (NCHCHOSi), 127.2, 127.6, 128.5 (3×aromatic CH), 138.1 (quaternary aromatic C), 168.0, 169.4, 170.3 (3×amide C═O); 7vDb: ¹H NMR (500 MHz, CD₃OD, COSY, nOe): δ=0.09 (s, 3H; SiCH₃), 0.15 (s, 9H; 3×SiCH₃), 0.90 (s, 18H; 2×SiC(CH₃)₃), 1.99 (s, 3H; CH₃CO), 3.48 (dd, ²J(H,H)=10.8 Hz, ³J(H,H)=2.0 Hz, 1H; NCHH′CHOSi), 3.88 (dd, ²J(H,H)=10.8 Hz, ³J(H,H)=3.9 Hz, 1H; NCHH′CHOSi), 3.99 (d, ²J(H,H)=16.7 Hz, 1H; CH₃CONHCHH′), 4.03 (d, ²J(H,H)=16.7 Hz, 1H; CH₃CONHCHH′), 4.23 (m, 1H; NCH₂CHOSi), 4.32 (m, 1H; NCHCHOSi), 4.34 (d, ²J(H,H)=15.5 Hz, 1H; NHCHH′Ph), 4.44 (d, ²J(H,H)=15.5 Hz, 1H; NHCHH′Ph), 4.54 (d, ³J(H,H)=5.2 Hz, 1H; NCHCHOSi), 7.19-7.35 (m, 5H; C₆H₅); ¹³C NMR (100 MHz, CDCl₃, HMQC): δ=−5.7, −5.6, −5.5 (Si(CH₃)₂), 17.7, 17.7 (SiC(CH₃)₂), 21.3 (CH₃CO), 25.2, 25.2 (SiC(CH₃)₃), 42.2 (CH₃CONCH₂), 43.2 (NCH₂Ph), 52.8 (NCH₂CHOSi), 65.2 (NCHCHOSi), 75.9 (NCH₂CHOSi), 77.0 (NCHCHOSi), 127.0, 127.4, 128.4 (aromatic CH), 138.6 (quaternary aromatic C), 169.5, 170.0, 172.7 (3×amide C═O); IR (film): ν̅=3065 (aromatic C—H), 2953, 2930, 2858 (aliphatic C—H), 1651 (3×amide C═O); MS (ES⁺): m/z (%): 586 (100) [M+Na]⁺, 564 (56) [M+H]⁺; HRMS (ES⁺): m/z: calcd for C₂₈H₅₀N₃O₅Si₂: 564.3289; found. 564.3309 [M+H]⁺.

### 1-(N-Acetyl-glycinyl)-2,3-trans-2-(benzylcarbamoyl)-3,4-trans-dihydroxy-pyrrolidine (8vD)

THF (1 mL) and trifluoroacetic acid (TFA) (aq., 50% v/v solution, 1 mL) were added to 1-(N-acetyl-glycinyl)-2,3-trans-2-(benzylcarbamoyl)-3,4-trans-O-di-tert-butyldimethylsilyl-pyrrolidine 6vD (30 mg, 0.053 mmol) and the reaction mixture was vigorously stirred under Ar. After 18 h TLC (EtOAc/petrol 4:1) showed consumption of SM (R_{f} 0.2) and formation of a major product (R_{f} 0.0). The mixture was concentrated under vacuum, then water (2×20 mL) was added and removed under vacuum to yield 8vD as a colourless oil (16 mg, 90%). ¹H NMR (400 MHz, CD₃OD): δ=2.01 (s, 3H; CH₃CO), 3.62 (d, ²J(H,H)=10.6 Hz, 1H; NCHH′CHOH), 3.90 (dd, ²J(H,H)=10.7 Hz, ³J(H,H)=4.7 Hz, 1H; NCHH′NCHOH), 4.00 (d, ²J(H,H)=17.1 Hz, 1H; CH₃CONCHH′), 4.11 (d, ²J(H,H)=17.1 Hz, 1H; CH₃CONCHH′), 4.18 (m, 1H; NCH₂CHOH), 4.25 (br s, 1H; NCHCHOH), 4.37 (d, ²J(H,H)=15.4 Hz, 1H; NCHH′Ph), 4.38 (m, 1H; NCH), 4.45 (d, ²J(H,H)=15.4 Hz, 1H; NCHH′Ph), 7.19-7.37 (m, 5H; C₆H₅); ¹³C NMR (125.7 MHz, CD₃OD, HMQC): δ=21.3 (CH₃CO), 41.9, 43.0 (2×NHCH₂), 52.7 (NCH₂), 68.6 (NCH), 75.4 (NCH₂CHOH), 78.4 (NCHCHOH), 126.9, 127.2, 128.4 (aromatic CH), 138.7 (quaternary aromatic C), 170.1, 171.0, 172.9 (3×amide C═O); IR (film): ν̅=3337 cm⁻¹ (br, O—H), 3067 (aromatic C—H), 2932 (aliphatic C—H), 1636 (3×amide C═O); MS (ES⁺): m/z (%): 358 (100) [M+Na]⁺, 336 (29) [M+H]⁺; HRMS (ES⁺): m/z: calcd for C₁₆H₂₁N₃O₅Na: 358.1379; found: 358.1362 [M+Na]⁺.

### 1-(N-Acetyl-glycinyl)-2,3-trans-2-benzylcarbamoyl-3,4-cis-dihydroxy-pyrrolidine (9vD)

THF (1 mL) and trifluoroacetic acid (TFA) (aq., 50% v/v solution, 1 mL) were added to 1-(N-acetyl-glycinyl)-2,3-trans-2-benzylcarbamoyl-3,4-cis-O-isopropylidene-pyrrolidine 7vD (67 mg, 0.120 mmol) and the reaction mixture vigorously stirred under Ar. After 45 h TLC (EtOAc) showed consumption of SM (R_{f} 0.2) and formation of a major product (R_{f} 0.0). The mixture was concentrated under vacuum, then water was added and removed under vacuum (2×20 mL) to yield 9vD as a colourless oil (25 mg, 62%). ¹H NMR (400 MHz, CD₃OD): δ=2.01 (s, 3H; CH₃CO), 3.53 (dd, ²J(H,H)=10.4 Hz, ³J(H,H)=5.6 Hz, 1H; NCHH′CHOH), 3.82 (dd, ²J(H,H)=10.4 Hz, ³J(H,H)=5.6 Hz, 1H; NCHH′CHOH), 4.01 (d, ²J(H,H)=17.0 Hz, 1H; CH₃CONHCHH′), 4.07 (d, ²J(H,H)=17.0 Hz, 1H; CH₃CONCHH′), 4.16 (pt, ³J(H,H)=4.2 Hz, 1H; NCHCHOH), 4.29 (m, 2H; NCH₂CHOH, NCH), 4.38 (d, ²J(H,H)=15.2 Hz, 1H; NCHH′Ph), 4.45 (d, ²J(H,H)=15.1 Hz, 1H; NCHH′Ph), 7.21-7.34 (m, 5H; C₆H₅); ¹³C NMR (125.7 MHz, CD₃OD, HMQC): δ=21.3 (CH₃CO), 41.5, 43.1 (2×NHCH₂), 50.6 (NCH₂), 66.6 (NCH), 70.8 (NCH₂CHOH), 75.0 (NCHCHOH), 127.1, 127.3, 128.5 (aromatic CH), 138.7 (quaternary aromatic C), 169.3, 171.4, 172.7 (3×amide C═O); lit (film): ν̅=3298 (br, O—H), 3096 (aromatic C—H), 2930 (aliphatic C—H), 1654 cm⁻¹ (3×amide C═O); MS (ES⁺): m/z (%): 358 (100) [M+Na]⁺, 336 (38) [M+H]⁺; HRMS (ES⁺): m/z: calcd for C₁₆H₂₁N₃O₅Na: 358.1379; found: 358.1371 [M+Na]⁺.

### 1,2:3,4-Diisopropylidene-α-D-galacturonic acid (x)ⁱ

Ruthenium trichloride hydrate (14.1 mg, 0.07 mmol, 0.02 eq) and sodium periodate (2.98 g, 13.9 mmol, 4.5 eq) were added to a solution of 1,2:3,4-diisopropylidene-α-D-galactose (800 mg, 3.07 mmol, 1.0 eq) in a mixture of CHCl₃ (32 mL), water (48 mL) and MeCN (32 mL) and the reaction mixture vigorously stirred under Ar. After 20 h stirring, TLC (EtOAc) indicated consumption of SM (R_{f} 0.7) and formation of a major product (R_{f} 0.1). The reaction mixture was diluted with CH₂Cl₂ (70 mL) and the organic layer separated. The aqueous phase was extracted with CH₂Cl₂ (2×70 mL) and the combined organic extracts were dried (MgSO₄) and concentrated to yield an oil. Purification by flash column chromatography on a short plug of silica gel (EtOAc) yielded x as a white solid (547 mg, 65%). m.p. 152-153° C. {lit.:^{3b} m.p. 157° C.; lit:.^{3a} m.p. 149-151° C.}; [α]_{D}²⁰=−86.0 (c=1.0 in CHCl₃) {lit.:^{3b} [α]_{D}=−92.0 (c=1.11 in CHCl₃)}; ¹H NMR (300 MHz, CDCl₃): δ=1.36 (m, 6H; C(CH₃)₂), 1.46, 1.54 (2×s, 2×3H; 2×C(CH₃)), 4.41 (dd, ³J(H,H)=4.9 Hz, ³J(H,H)=2.6 Hz, 1H; H-2), 4.47 (d, ³J(H,H)=1.9 Hz, 1H; H-5), 4.62-4.71 (m, 2H; H-3, H-4), 5.66 (d, ³J(H,H)=4.9 Hz, 1H; H-1), 6.74 (br s, 1H; CO₂H); MS (APCI⁻): m/z (%): 273 (100) [M−H]⁻.

### 1-Butanoyl-2,3-trans-2-(1-pentylcarbamoyl)-3,4-cis-O-isopropylidene-pyrrolidine (6iE)

DBU (635 μL, 646 mg, 4.24 mmol, 1.3 eq) was added to N-chloro-2,3-O-isopropylidene-1,4-dideoxy-1,4-iminoerythritol 1 (580 mg, 3.27 mmol, 1.0 eq) in dry THF (12 mL) under Ar. After 3.5 h stirring, the mixture was filtered to remove DBU.HCl and concentrated under reduced pressure. The residue was dissolved in dry MeOH (6 mL) and butyric acid (389 μL, 375 mg, 4.24 mmol, 1.3 eq) and 1-pentyl isocyanide (527 μL, 413 mg, 4.24 mmol, 1.3 eq) were added. After 18 h stirring, the mixture was concentrated to dryness, then dissolved in EtOAc (40 mL) and washed with NH₄Cl (aq.) (10 mL) followed by NaHCO₃ (aq.) (10 mL) The organic layer was dried (Na₂SO₄) and concentrated to yield 6iE as an orange oil (705 mg, 66%). ¹H NMR (300 MHz, CDCl₃, COSY, nOesy): δ=0.81 (t, ³J(H,H)=6.6 Hz, 3H; CONH(CH₂)₄CH₃), 0.90 (t, ³J(H,H)=7.4 Hz, 3H; CO(CH₂)₂CH₃), 1.22 (m, 4H; CONHCH₂CH₂(CH₂)₂CH₃), 1.25, 1.35 (2×s, 2×3H; C(CH₃)₂), 1.40 (m, 2H; CONHCH₂CH₂), 1.60 (in, 2H; COCH₂CH₂), 2.24 (m, 2H; COCH₂), 3.12 (m, 2H; CONHCH₂), 3.57 (dd, ²J(H,H)=12.0 Hz, ³J(H,H)=4.9 Hz, 1H; NCHH′), 3.71 (d, ²J(H,H)=12.0 Hz, 1H; NCHH′), 4.69 (s, 1H; NCHCONH), 4.81 (m, 1H; NCH₂CHO), 5.02 (d, ³J(H,H)=6.0 Hz, 1H; NCHCHO); ¹³C NMR (75 MHz, CDCl₃, HMQC): δ=11.6, 11.8, 16.2, 20.1, 22.6, 24.6, 26.8, 26.9 (8×C; COCH₂CH₂CH₃, CONHCH₂(CH₂)₃CH₃, C(CH₃)₂), 34.1 (COCH₂), 37.2 (CONHCH₂), 51.0 (NCH₂), 63.1 (NCHCONH), 77.5 (NCH₂CHO), 82.0 (NCHCHO), 109.6 (C(CH₃)₂), 167.0, 170.9 (CONHCH₂, NCOCH₂); IR (film): ν̅=2960, 2934, 2874 (aliphatic C—H), 1651 cm⁻¹ (2×NH(C═O)); HRMS (ES⁺): m/z: calcd for C₁₇H₃₁N₂O₄: 327.2284; found: 327.2281 [M+H]⁺.

### 1-Butyl-2,3-trans-2-(1-pentylcarbamoyl)-3,4-cis-O-isopropylidene-pyrrolidine (10)

### 2,3-trans-2-(1-Pentylcarbamoyl)-3,4-cis-O-isopropylidene-pyrrolidine (11)

Lithium aluminum hydride (powder, 32 mg, 0.833 mmol, 1.0 eq) was added to 1-butanoyl-2,3-trans-2-(1-pentylcarbamoyl)-3,4-cis-O-isopropylidene-pyrrolidine 9iE in dry THF (10 mL) under Ar. After 1 h stirring TLC (EtOAc/petrol 4:1) showed that mainly SM (R_{f} 0.5) remained. A further portion of lithium aluminum hydride (16 mg, 0.5 eq) was added and after a further 1 h, TLC showed consumption of SM and the formation of two major products (R_{f} 0.6 and R_{f} 0.2). The reaction mixture was filtered through a plug of silica gel then concentrated under reduced pressure.

The residue was purified by flash column chromatography on silica gel (EtOAc/petrol 1:1) whereupon three products were recovered, all as colourless oils: 10 (85 mg, 33%), unreacted SM 9iE (14 mg, 5%) and 11 (80 mg, 37%). 10: ¹H NMR (300 MHz, CDCl₃, COSY, nOesy): δ=0.82 (t, ³J(H,H)=7.1 Hz, 3H; CONH(CH₂)₄CH₃), 0.85 (t, ³J(H,H)=7.4 Hz, 3H; N(CH₂)₃CH₃), 1.20-1.43 (m, 10H; CONHCH₂(CH₂)₃CH₃, NCH₂(CH₂)₂CH₃), 1.23, 1.45 (2×s, 2×3H; C(CH₃)₂), 2.61, 2.73 (2×m, 2×1H; NCH₂(CH₂)₂CH₃), 2.71 (dd, ²J(H,H)=11.8 Hz, ³J(H,H)=3.4 Hz, 1H; NCHH′), 3.11 (dd, ²J(H,H)=11.8 Hz, ³J(H,H)=5.4 Hz, 1H; NCHH′), 3.18 (m, 2H; CONHCH₂), 3.34 (d, ³J(H,H)=1.9 Hz, 1H; NCHCONH), 4.57 (ptd, ³J(H,H)=5.6 Hz, ³J(H,H)=3.4 Hz, 1H; NCH₂CHO), 4.72 (dd, ³J(H,H)=6.0 Hz, ³J(H,H)=1.9 Hz, 1H; NCHCHO); ¹³C NMR (75 MHz, CDCl₃, HMQC, HMBC): δ=14.3, 14.3 (CONHCH₂(CH₂)₃CH₃, N(CH₂)₃CH₃), 20.7, 22.7, 24.9, 27.4, 29.5, 29.6, 31.8 (7×C; NCH₂(CH₂)₂CH₃, CONHCH₂(CH₂)₃CH₃, C(CH₃)₂), 39.3 (CONHCH₂), 56.5 (NCH₂(CH₂)₂CH₃), 58.5 (NCH₂), 73.8 (NCHCONH), 80.5 (NCH₂CHO), 84.8 (NCHCHO), 112.9 (C(CH₃)₂), 171.1 (CONHCH₂); 1R (film): ν̅=2958, 2933, 2861 (aliphatic C—H), 1651 cm⁻¹ (NH(C═O)); HRMS (ES⁺): m/z: calcd for C₁₇H₃₃N₂O₃: 313.2491; found: 313.2492 [M+H]⁺. 11: ¹H NMR (300 MHz, CDCl₃, COSY, nOesy): δ=0.82 (t, ³J(H,H)=6.8 Hz, 3H; CONH(CH₂)₄CH₃), 1.23 (m, 4H; CONHCH₂CH₂(CH₂)₂CH₃), 1.24, 1.38 (2×s, 2×3H; C(CH₃)₂), 1.42 (m, 2H; CONHCH₂CH₂), 2.52 (dd, ²J(H,H)=13.9 Hz, ³J(H,H)=3.8 Hz, 1H; NCHH′), 3.02 (d, ²J(H,H)=13.9 Hz, 1H; NCHH′), 3.14 (m, 2H; CONHCH₂), 3.62 (s, 1H; NCHCONH), 4.56 (m, 1H; NCH₂CHO), 5.12 (d, ³J(H,H)=5.7 Hz, 1H; NCHCHO); ¹³C NMR (75 MHz, CDCl₃, HMQC, HMBC): δ=14.3 (CONHCH₂(CH₂)₃CH₃), 22.7, 24.2, 26.5, 29.4, 29.6 (5×C; CONHCH₂(CH₂)₃CH₃, C(CH₃)₂), 39.4 (CONHCH₂), 53.0 (NCH₂), 68.7 (NCHCONH), 82.1 (NCH₂CHO), 84.4 (NCHCHO), 111.1 (C(CH₃)₂), 169.9 (CONHCH₂); IR (film): ν̅=2960, 2865 (aliphatic C—H), 1652 cm⁻¹ (NH(C═O)); HRMS (ES⁺): m/z: calcd for C₁₃H₂₅N₂O₃: 257.1865; found: 257.1859 [M+H]⁺.

### 1-Butyl-2,3-trans-2-(1-pentylcarbamoyl)-3,4-cis-dihydroxy-pyrrolidine trifluoroacetate (12)

TFA (50% v/v aq. solution, 1 mL) and THF (1 mL) were added to 1-butyl-2,3-trans-2-(1-pentylcarbamoyl)-3,4-cis-O-isopropylidene-pyrrolidine 10 (75 mg, 0.24 mmol, 1.0 eq) and the reaction mixture stirred vigorously. After 70 h, TLC (EtOAc) showed that SM remained (R_{f} 0.6), so the reaction mixture was heated to 50° C. After 20 h at this temperature, TLC revealed consumption of SM and formation of a major product (R_{f} 0.05). The reaction mixture was concentrated to dryness, then lyophilised to yield 12 as an oil (81 mg, 87%). ¹H NMR (300 MHz, D₂O, COSY, nOesy): δ=0.72 (t, ³J(H,H)=6.8 Hz, 3H; CONH(CH₂)₄CH₃), 0.75 (t, ³J(H,H)=7.5 Hz, 3H; N(CH₂)₃CH₃), 1.12-1.48 (m, 10H; CONHCH₂(CH₂)₃CH₃, NCH₂(CH₂)₂CH₃), 3.04-3.24 (m, 5H; NCH₂(CH₂)₂CH₃, CONHCH₂, NCHH′), 3.84 (dd, ²J(H,H)=13.2 Hz, ³J(H,H)=4.2 Hz, 1H; NCHH′), 3.97 (d, ³J(H,H)=9.0 Hz, 1H; NCHCONH), 4.15 (dd, ³J(H,H)=9.0 Hz, ³J(H,H)=4.0 Hz, 1H; NCHCHO), 4.25 (m, 1H; NCH₂CHO); ¹³C NMR (75 MHz, D₂O, HMQC): δ=10.9, 11.4 (CONHCH₂(CH₂)₃CH₃, N(CH₂)₃CH₃), 17.1, 19.7, 25.0, 26.0, 26.4 (5×C; NCH₂(CH₂)₂CH₃, CONHCH₂(CH₂)₃CH₃), 38.0 (CONHCH₂), 56.9, 57.5 (NCH₂(CH₂)₂CH₃, NCH₂), 67.1, 67.6 (NCHCONH, NCH₂CHO) 72.5 (NCHCHO), 114.3 (q, ^{I}J(C,F)=289.3 Hz, CF₃CO₂⁻), 160.9 (q, ²J(C,F)=35.8 Hz, CF₃CO₂⁻), 164.8 (CONHCH₂); IR (film): ν̅=3348 (br, O—H), 2954, 2932, 2863 (aliphatic C—H), 1653 cm⁻¹ (NH(C═O)); HRMS (ES⁺): calcd for C₁₄H₂₉N₂O₃: 273.2178; found: 273.2168 [M+H]⁺.

### 2-(1-Pentylcarbamoyl)-3,4-cis-dihydroxy-pyrrolidine trifluoroacetate (13)

TFA (50% v/v aq. solution, 1 mL) and THF (1 mL) were added to 2,3-trans-2-(1-pentylcarbamoyl)-3,4-cis-O-isopropylidene-pyrrolidine 11 (70 mg, 0.27 mmol, 1.0 eq) and the reaction mixture stirred vigorously. After 70 h, TLC (EtOAc) showed that SM remained (R_{f} 0.2), so the reaction mixture was heated to 50° C. After 20 h at this temperature, TLC revealed consumption of SM and formation of a major product (R_{f} 0.05). The reaction mixture was concentrated to dryness, then lyophilised to yield 13 as an oily solid (81 mg, 90%). ¹H NMR (300 MHz, D₂O, COSY, nOesy): δ=0.71 (t, ³J(H,H)=6.8 Hz, 3H; CONH(CH₂)₄CH₃), 1.15 (m, 4H; CONH(CH₂)₂(CH₂)₂CH₃), 1.39 (m, 2H; CONHCH₂CH₂), 3.12 (n, 2H; CONHCH₂), 3.28 (dd, ²J(H,H)=12.8 Hz, ³J(H,H)=2.2 Hz, 1H; NCHH′), 3.45 (d, ²J(H,H)=12.8 Hz, ³J(H,H)=4.1 Hz, 1H; NCHH′), 3.98 (d, ³J(H,H)=7.9 Hz, 1H; NCHCONH), 4.19 (dd, ³J(H,H)=7.9 Hz, ³J(H,H)=3.8 Hz, 1H; NCHCHO), 4.25 (m, 1H; NCH₂CHO); ¹³C NMR. (75 MHz, D₂O, HMQC): δ=12.2 (CONHCH₂(CH₂)₃CH₃), 20.6, 26.8, 27.2 (3×C; CONHCH₂(CH₂)₃CH₃), 38.9 (CONHCH₂), 49.1 (NCH₂), 60.3 (NCHCONH), 68.9 (NCH₂CHO), 73.9 (NCHCHO), 115.2 (q, ¹J(C,F)=289.3 Hz, CF₃CO₂⁻), 161.8 (q, ²J(C,F)=35.6 Hz, CF₃CO₂⁻), 166.3 (CONHCH₂); IR (film): ν̅=3362 (hr, O—H, N—H), 2962, 2867 (aliphatic C—H), 1656 cm⁻¹ (NH(C═O)); HRMS (ES⁺): m/z: calcd for C₁₀H₂₁N₂O₃: 216.1474; found: 216.1478 [M+H]⁺.

General Experimental for Library Work

All products were analysed by ¹H NMR and LC-MS. ¹H NMR spectra were recorded on a Bruker AV300 spectrometer at 300 MHz. ¹H NMR spectra on all Ugi products were run in deuterated chloroform, and on deprotected products in d₄-methanol. Mass spectra were recorded on a Micromass ZMD 2000 mass spectrometer using APCI ionisation; all samples were pre-run through a Waters 2690 HPLC with a Waters symmetry packed LC column (C8 dimethyloctylsilyl bonded amorphous silica, 3.5 μm, 4.6×50 mm, over a 5 min run, starting with H₂O/MeOH 4:1 and increasing to 100% MeOH after 3 mins; retention times are quoted below).

Library Approach, Typical Procedure:

Ugi-Type Reaction, Erythritol Imine 3, n-butyl Isocyanide Series, 6(i-ix)C: DBU

(0.724 mL, 737 mg, 4.84 mmol, 14.3 eq) was added to a solution of N-chloro-2,3-O-isopropylidene-1,4-dideoxy-1,4-iminoerythritol 1 (660 mg, 3.72 mmol, 11.0 eq) in dry THF (18 mL) under Ar. The reaction mixture was stirred for 3.5 h then the mixture was filtered to remove DBU.HCl. The filtrate was concentrated under reduced pressure to yield the erythritol imine 3 as an oil. This was dissolved in dry MeOH (11 mL) and a portion (1.0 mL) added to each of 11 tubes. The relevant carboxylic acid (i-ix, Table 1, 0.439 mmol, 1.3 eq) and n-butyl isocyanide (46 μL, 36 mg, 0.439 mmol, 1.3 eq) were added to each tube. After 18 h shaking the reaction mixtures were concentrated to dryness and partitioned between EtOAc (5 mL) and NaHCO₃ (aq.) (2 mL) The organic layer was dried (Na₂SO₄) and concentrated under reduced pressure to yield the Joullié-Ugi products. For yields see table 4 and for characterisation data see table 5.

Ugi-Type Reaction, Threitol Imine 4, n-butyl Isocyanide Series, 7(i-ix)C: DBU

(0.657 mL, 669 mg, 2.34 mmol, 33.0 eq) was added to a solution of N-chloro-2,3-O-tert-butyldimethylsilyl-1,4-dideoxy-1,4-iminothreitol 2 (660 mg, 1.80 mmol, 11.0 eq) in dry THF (18 mL) under Ar. The reaction mixture was stirred for 3.5 h then the mixture was filtered to remove DBU.HCl. The filtrate was concentrated under reduced pressure to yield the threitol imine 4 as an oil. This was dissolved in dry MeOH (11 mL) and a portion (1.0 mL) added to each of 11 tubes. The relevant carboxylic acid (i-ix, Table 1, 0.492 mmol, 3.0 eq) and n-butyl isocyanide (51 μL, 41 mg, 0.492 mmol, 3.0 eq) were added to each tube. After 18 h shaking the reaction mixtures were concentrated to dryness and partitioned between EtOAc (5 mL) and NaHCO₃ (aq.) (2 mL) The organic layer was dried (Na₂SO₄) and concentrated under reduced pressure to yield the Ugi-type products.

Deprotection: THF (1 mL) and TFA (50% v/v aq. solution, 1 mL) were added to each of the Ugi-type products in tubes. After 18 h shaking, the reaction mixtures were concentrated to dryness then lyophilised to afford the deprotected Ugi-type products. In the case of the threitol imine Ugi-type products, reaction mixtures were shaken for 42 h.

Ugi-Type Products in Reaction of Erythritol Imine 3 with more Complex Substrates:

DBU (66 μL, 67 mg, 0.44 mmol, 1.3 eq) was added to a solution of N-chloro-2,3-O-isopropylidene-1,4-dideoxy-1,4-iminoerythritol 1 (60 mg, 0.34 mmol, 1.0 eq) in dry THE (2 mL) under Ar. The reaction mixture was stirred for 3.5 h then the mixture was filtered to remove DBU.HCl. The filtrate was concentrated under reduced pressure to yield the erythritol imine 3 as an oil. This was dissolved in dry MeOH (1.0 mL) under Ar and the relevant carboxylic acid (0.44 mmol, 1.3 eq) and isocyanide (0.44 mmol, 1.3 eq) were added. After 18 h shaking the reaction mixture was concentrated to dryness and partitioned between EtOAc (5 mL) and NaHCO₃ (aq.) (2 mL). The organic layer was dried (Na₂SO₄) and concentrated under reduced pressure to yield the Uri-type product.

Further Experimental Details of Library Preparation

The use of either anhydrous DCM or methanol as solvent gives similarly good yields and diastereoselectivites, but in order to promote the use of the Ugi reaction as a tool for library development we investigated the use of more convenient HPLC methanol rather than stringently anhydrous methanol. Although the reaction proceeded well, the product was not as pure after partitioning between DCM and sodium bicarbonate (presumably in some part due to imine hydrolysis and to the presence of some unreacted isocyanide not removed during the aqueous washes). Neither an acidic nor a brine wash were successful in removing the isocyanide. Although treatment of this crude mixture with TFA both deprotected the product and hydrolysed the isocyanide allowing facile purification by reverse phase chromatography. The preferred treatment used was anhydrous methanol.

Deprotection of 7 with tetrabutylammonium fluoride led to the same products as TFA, yet the long term need for ease of purification in library format meant that the TFA method was preferred.

In certain instances MCR yields were below what may have been expected from literature examplesⁱⁱ e.g. 43-77% for reaction of 1, v and varying isocyanides. Although these are acceptable yields, it may be that some product is lost to the aqueous wash during work up.

Several alternative isocyanides and carboxylic acids were considered for inclusion in the library: a complex mixture of side products was observed when 2-morpholinyethyl isocyanide was used,ⁱⁱⁱ likewise for N-methylpyrrole-2-carboxylic acid. 2-Ethylhexanoic acid led to the desired Ugi product, but with a relatively high level of impurity. Threitol products 9iiiX were observed but since a three-fold excess of acid (c.f. 1.3 equivalents for erythritol imines) was used, reverse phase chromatography was required for its separation from the bisamide product. These difficulties in purification led to their omission from the library.

Enzyme Inhibition Assays

Human Glycosidases and Glucosylceramide Synthase

Enzymes were extracted from an MCF7 cell-line harvest: cells were harvested from ≈100 mL of standard culture, washed in phosphate buffered saline solution (PBS) and sonicated (3×10 s) in water (1 mL). Extract (5 μL) and inhibitor solution (0.04, 0.4 or 4 mM diluted using water from 100 mM stock in DMSO, 5 μL) were diluted with the appropriate enzyme assay solution (see below, 10 μL) and incubated for the appropriate length of time (see below). The course of the assay was stopped by addition of glycine-carbonate buffer solution (0.17 M, pH 9.8, 150 μL) and absorbance (405 nm) or fluorescence (excitation 460 nm, emission 355 nm) recorded as appropriate. Assay solutions and incubation times: α-D-glucosidase [1.25 mM p-nitrophenyl α-D-glucopyranoside in 0.2 M citrate/phosphate buffer, pH 4.4, 37° C., 16 h]; β-D-glucosidase [5 mM 4-methylumbelliferyl β-D-glucopyranosid.e in 0.2 M citrate/phosphate buffer, pH 5.8, 37° C., 3 H]; α-D-galactosidase [20 mM p-nitrophenyl α-D-galactopyranoside, 180 mM N-acetyl-D-glucosamine in 0.2 M citrate/phosphate buffer, pH 4.4, 37° C., 4 h]; β-D-galactosidase [5 mM p-nitrophenyl β-D-galactopyranoside in 0.2 M citrate/phosphate buffer, pH 4.3, 37° C., 2 h]; α-D-mannosidase [8 mM p-nitrophenyl α-D-mannopyranoside in 0.2 M citrate/phosphate buffer, pH 4.4, 37° C., 1.5 h].

Glucosylceramide synthase [UDP-glucose N-acyl-sphingosine glucosyltransferase (EC 2.4.1.80)] assay was conducted using HL-60 cell microsomes as described in the literature.^{iv}

All inhibitors were screened at a concentration of 100 μM against glycosidases and 50 μM against glucosylceramide synthase; all assays were recorded in duplicate.

Non-Mammalian Glycosidases

p-Nitrophenyl-glycosides were purchased from Sigma-Aldrich Co. Ltd. Enzymes were purchased from Sigma-Aldrich: α-mannosidase (Canavalia ensiformis, jack beans, M7257), β-mannosidase (snail acetone powder, M9400), β-glucosidase (almonds, G0395), α-galactosidase (green coffee beans, G8507) and α-rhamnosidase (Penicillium decumbens, naringinase, N1385). Enzyme solutions (0.1 U mL⁻¹ in appropriate buffer (see below), 5 μL) and inhibitor solutions (1 mM diluted using water from 10 mM stock in DMSO, 5 μL) were diluted with the appropriate enzyme assay solution (see below, 40 μL) and incubated for 1 hour. The course of the assay was stopped by addition of glycine-carbonate buffer solution (0.17 M, pH 9.8, 150 μL) and the absorbance (405 nm) recorded. Assay solutions: α-D-mannosidase [4.0 mM p-nitrophenyl α-D-mannopyranoside in 0.2 M citrate/phosphate buffer, pH 4.5, 37° C.]; β-D-mannosidase [0.8 mM p-nitrophenyl β-D-mannopyranoside in 0.2 M citrate/phosphate buffer, pH 4.0, 37° C.]; β-D-glucosidase [2.0 mM p-nitrophenyl β-D-glucopyranoside in 0.2 M citrate/phosphate buffer, pH 5.0, 37° C.]; α-D-galactosidase [2.0 mM p-nitrophenyl α-D-galactopyranoside, pH 6.5, 37° C.]; naringinase [1.0 mM p-nitrophenyl α-L-rhamnopyranoside in 0.2 M citrate/phosphate buffer, pH 4.0, 37° C.]. All assays were recorded in duplicate.

Factor Inhibiting Hypoxia-Inducing Factor and PHD2

Compounds were tested for inhibitory potential against two iron(II) and 2-oxoglutarate (2OG) dependent dioxygenases that function as part of the hypoxic response in humans. PHD2 is one of three isozymes that can hydroxylate hypoxia-inducible factor α (HIFα) under normoxic conditions at conserved prolyl residues in the oxygen dependent degradation domains of HIF (Pro-402 and Pro-564 in human HIF-1α)^{va}. This modification allows binding to the von Hippel-Lindau-Elongin C-Elongin B complex which in turn allows recognition by E3 ubiquitin ligase, subsequent ubiquitination and degradation in the proteasome.^{5b,c} Under hypoxic conditions, HIF-1α can dimerise with its partner HIF-1β (also known as ARNT), and bind to hypoxia response elements in the upstream region of genes such as erythropoietin and vascular endothelial growth factor.^{5d} In a second control system, FIH can hydroxylate HIFα on the β-position^{vi} of a conserved asparaginyl residue (Asn-803 in human HIF-1α) in the C-terminal activation domain of HIF. This modification prevents association with p300, an interaction that is necessary to activate transcription of the genes mentioned above.^{vii} Thus, in hypoxia, the hydroxylation does not occur and transcriptional activation is possible.

The assay used follows the consumption of 2OG by the enzymes by the use of a post-reaction derivatisation of the remaining 2OG with o-phenylene diamine to form a fluorescent product. Essentially, a reaction mixture comprising 1 mM DTT, 0.6 mg/ml catalase, 500 μM 2OG, 800 μM synthetic peptide corresponding to the CAD region of HIF for FIH or 100 μM synthetic 19 mer peptide for PHD2, 1 mM test compound and 50 mM Tris/HCl pH 7.5 with 4 μM FIH and 50 μM iron(II) was incubated for 5 minutes, whereupon the reaction was stopped and the remaining 2OG detected (Details are given in McNeill et al 2004, submitted).

Porcine Pancreatic Elastatse

Succinyl-Ala-Ala-Pro-Ala-p-nitroanilide was purchased from Bachem, porcine pancreatic elastase was purchased from Serve. Electrophoresis. PPE (0.02 μM in TRIS.HCl buffer) was added to substrate solution (0.3 mM in buffer), inhibitor solution (3.0 mM in buffer), DMSO (up to 10% by volume) in a total volume of 200 μM. Absorbance (405 nm) was recorded every 7 seconds for 5 minutes at 23° C.

Bovine Viral Diarrhoea Virus

Madin-Darby bovine kidney cells (MDBK) were grown in DMEM/F12 (purchased from Gibco BRL) supplemented with 10% heat-inactivated horse serum (Gibco BRL). The cytopathic NADL strain of bovine viral diarrhoea virus (BVDV) (kindly provided by Dr. Ruben Donis, University of Nebraska) stock was prepared as previously described.^{viii} For antiviral activity testing MDBK cells were plated into 24 well plates 24 hours before virus infection. Cells were infected with BVDV at moi=0.5 for 1 hour in 100 μL media with occasional rocking of the plate. The inocula were washed twice with media and regular media were added with 100 μM of the test compound. After 24 hours, the supernatant media were saved. Secreted viruses in the media were titred on new MDBK cells by standard plaque assay. Toxicity of the compounds was determined by examining the tissue culture cells after the 24 hour treatment.

Characterisation of Library Products:

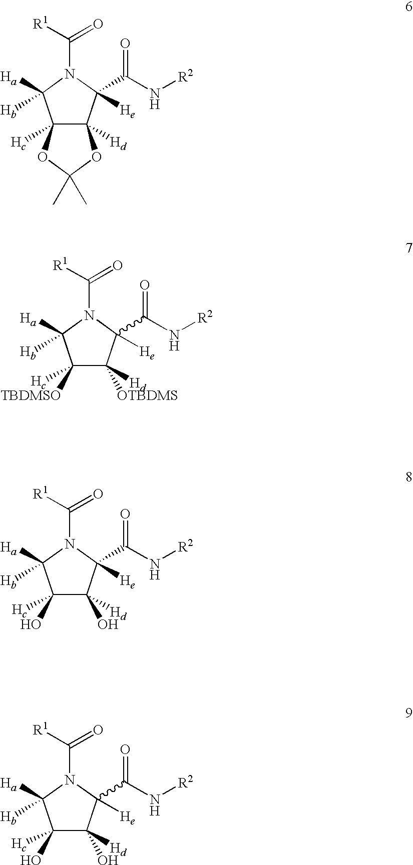

Key to ¹H NMR assignments

TABLE 1
Yields of 6 from Joullié-Ugi reaction of erythro substrate 1.

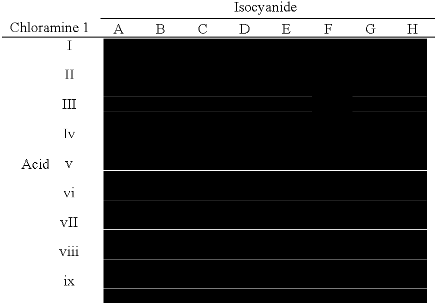

| | |
|---|---|
| 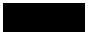 | Yields refer to Joullié-Ugi product from N-chloramine |
| 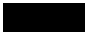 | Deprotection Yields |

Isocyanides: A = 1,1,3,3-Tetramethylbutyl; B = Cyclohexyl; C = n-Butyl;
D = Benzyl; E = 1-Pentyl; F = Isopropyl; G = 2-Pentyl; H = tert-Butyl.
Acids: I = Butyric; ii = Benzoic; iii = 5-Hexenoic; iv = 2-(2-Methoxyeth-
oxy)acetic acid; v = N-Ac-glycine; vi = 2,4-Difluorobenzoic; vii = 3-Di-
methylaminobenzoic; viii = 3-Furoic; Ix = Nicotinic.

TABLE 2
Yields of 7 from Joullié-Ugi reaction of threo substrate 2.

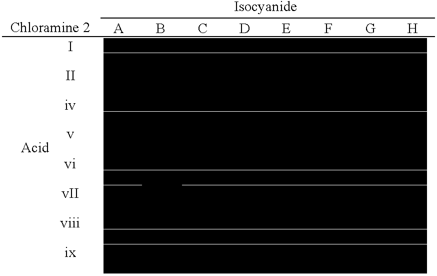

| | |
|---|---|
| 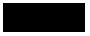 | Yields refer to Joullié-Ugi product from N-chloramine |
| 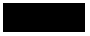 | Deprotection Yields |

Isocyanides: A = 1,1,3,3-Tetramethylbutyl; B = Cyclohexyl; C = n-Butyl;
D = Benzyl; E = 1-Pentyl; F = Isopropyl; G = 2-Pentyl; H = tert-Butyl.
Acids: i = Butyric; ii = Benzoic; iv = 2-(2-Methoxyethoxy)acetic acid;
v = N-Ac-glycine; vi = 2,4-Difluorobenzoic; vii = 3-Dimethylamino-
benzoic; viii = 3-Furoic; ix = Nicotinic.

TABLE 3
¹H NMR assignment and mass spectrometry of complex acid and
isocyanide substrates.

| | | Ring H | | |
|---|---|---|---|---|
| Isocyanide | Acid | (Hₐ-Hₑ) | (CH₃)₂ | R¹ |
| 1-Pentyl | 1,2:3,4- | 2.56 (m, 1H; NCHH′), | 1.24-1.65 (m, 18H; 3 × C(CH₃)₂) | 3.46-4.78 (m, 4H; H-2′, H- |
| isocyanide E | Diisopropylidene | 3.06-3.20 (m, 1H; NCHH′), 4.50-4.78 (m, | | 5′, H-3′, H-4′), 5.50 (d, |
| | galacturonic acid x | 1H; CHCONH), 4.85 (m, 1H; | | ³J (H, H) = 4.9 Hz, 0.5H; H- |
| | | NCHCHO), 5.01 (m, 1H; | | 1′), 5.57 (d, ³J (H, H) = 4.9 Hz, |
| | | NCH₂CHO) | | 0.5H; H-1′) |
| α-(S)- | 3-Furoic acid xI | 3.52-3.98 (m, 2H; NCH₂), | 1.22-1.39 (m, 6H; | 6.57, 6.63, 7.28, 7.32, |
| Methylbenzyl | | 4.80-5.09 (m, 3H; NCHCONH, | C(CH₃)₂) | 7.65, 7.74 (6 × s, 6 × 0.5H; |
| isocyanide I | | NCH₂CH, NCHCH) | | 3H furan) |
| 1-Pentyl | N-Acetyl-L- | 1.16-1.48 (m, 6H; C(CH₃)₂), | 1.25, | 1.84 (s, 1.5H; CH₃CO), |
| isocyanide E | phenylalanine xII | 3.56-3.86 (m, 3H; NCH₂), 4.64-4.76 (m, | 1.33 (m, 6H; | 1.92 (s, 1.5H; CH₃CO), |
| | | 2H; NCH₂CH, NCH CONH), | C(CH₃)₂) | 3.09 (m, 2H; CH(CH₂Ph), |
| | | 4.90 (d, 0.5H; NCHCH), 4.96 (d, 0.5H; | | 4.72 (m, 1H; CH(CH₂)Ph), |
| | | NCHCH) | | 7.03-7.27 (m, 5H; C₆H₅) |

| | | m/z | | | |
|---|---|---|---|---|---|
| Isocyanide | R² | [Ion] | R₁/mln | Yield^{[a]} & dr^{[b]} | Product |
| 1-Pentyl | 0.84 (m, 3H; (CH₂)₄CH₃), | 513 [M + H]⁺ | 2.60 | 44% | 6xE |
| isocyanide E | 1.24-1.65 (m, 6H; | | | (1:1) | |
| | NCH₂(CH₂)₃CH₃), 3.32 (m, | | | | |
| | 2H; CONHCH₂) | | | | |
| α-(S)- | 1.58 (m, 3H; | 385 [M + H]⁺ | 2.92 | 51% | 6xII |
| Methylbenzyl | NCH(CH₃)Ph); 4.71 (m, | | | (1:1) | |
| isocyanide I | 1H; NCH(CH₃)Ph), | | | | |
| | 7.26 (m, 5H; C₆H₅) | | | | |
| 1-Pentyl | 0.76-0.87 (m, 3H; | 446 [M + H]⁺ | 3.20 | 59% | 6xIIE |
| isocyanide E | N(CH₂)₄CH₃), | | | (1:1) | |
| | 1.16-1.48 (m, 6H; | | | | |
| | CONHCH₂(CH₂)₃CH₃), | | | | |
| | 3.30 (m, 2H; CONHCH₂) | | | | |

¹H NMR and Mass Spec Characterisation Data: Ugi-type Products from Reaction of Erythritol Imine3 with-More Complex Substrates
1-(1,2:3,4-Diisopropylidene-α-D-galacturonyl)-2,3-trens-2-(1-pentylcarbamoyl)-3,4-cis-O-Isopropylidene-pyrrolidine (6xE)
1-(3-Furoyl)-2,3-trans-2-(α-(S)-methylbenzylcarbamoyl)-3,4-cis-O-isopropylidene-pyrrolidine (6xII)
1-(N-Acetyl-L-phenylalaninyl)-2,3-trans-2-(1-pentylcarbamoyl)-3,4-cis-O-isopropylidene-pyrrolidine (6xIIE)
^{[a]}Yields over 2-step sequence from N-chloramine 1;
^{[b]}Determined by ¹H NMR
6xII ¹³C NMR (75 MHz, CDCl₃, HMQC): δ = 20.8, 21.2, 23.5, 23.5, 25.5, 25.5 (3 × C(CH₃)₂, NHCH(CH₃)Ph), 47.8, 47.9 (NHCH(CH₃)Ph), 52.7, 53.0 (NCH₂), 64.4, 64.8 (NCHCONH), 78.4, 78.5. 78.7, 79.0 (NCHCHO, NCH₂CHO), 108.9, 109.0, 110.6, 110.7, 119.8, 119.9 (C(CH₃)₂, 2 × C furan), 124.3, 124.7, 125.7, 126.0, 127.2, 127.3 (aromatic CH), 141.6, 141.7, 142.1, 142.8, 143.2, 143.3 (quaternary aromatic C, 2 × C furan), 155.0, 155.1, 166.6, 166.9 (2 × amide C═O).

>1500 screens of library members against a) α-D-glucosidase, human; b) β-D-glucosidase, human; c) α-D-galactosidase, human; d) β-D-galactosidase, human; e) α-D-mannosidase, human; f) a-D-galactosidase, non-mammalian; g) β-D-glucosidase, non-mammalian; h) α-D-mannosidase, non-mammalian; i) β-D-mannosidase, non-mammalian; j) α-L-rhamnosidase, non-mammalian; k) GCS, human; l PPE; m) PHD2; n) FIH1; o) BVDV.

ⁱa) S. Torii, T. Inokuchi, T. Suguira, J. Org. Chem. 1986, 51, 155; b) H. M. Sell, K. P. Link, J. Am. Chem. Soc. 1938, 60, 1813.

ⁱⁱS. Marcaccini, D. Miguel, T. Torroba, M. Garcia-Valverde, J. Org. Chem. 2004, 68, 3315

ⁱⁱⁱZ. Li, S. L. Yea, C. J. Pallen, A. Ganesan, Bioorg. Med. Chem. Lett. 1998, 8, 2443.

^{iv}F. M. Platt, G. R. Neises, R. A. Dwek, T. D. Butters, J. Biol. Chem. 1994, 269, 8362.

^{v}a) A. C. R. Epstein, J. M. Gleadle, L. A. McNeill, K. S. Hewitson, J. O'Rourke, D. R. Mole, M. Mukherji, E. Matzen, M. I. Wilson, A. Dhanda, Y-M. Tian, N. Masson, D. L. Hamilton, P. Jaakola, R. Barstead, J. Hodgkin, P. H. Maxwell, C. W. Pugh, C. J. Schofield, P. J. Ratcliffe, Cell 2001, 107, 43; b) P. Jaakkola, D. R. Mole, Y-M. Tian, M. I. Wilson, J. Gielbert, S. J. Gaskell, A. van Kriegsheim, H. F. Hebestreit, M. Mukherji, C. J. Schofield, P. H. Maxwell, C. W. Pugh, P. I. Ratcliffe, Science 2001, 292, 468; c) M. Ivan, K. Kondo, H. Yang, W. Kim, J. Valiando, M. Ohh, A. Salic, J. M. Asara, W. S. Lane, W. G. Kaelin Jr., Science 2001, 292, 464; d) C. J. Schofield, P. J. Ratcliffe, Nat. Rev. Mol. Cell. Biol. 2004, 5, 343.

^{vi}See L. A. McNeill, K. S. Hewitson, T. D. Claridige, J. F. Siebel, L. E. Horsfall, C. J. Schofield, Biochem. J 2002, 367, 571.

^{vii}D. Lando, D. J. Peet, D. A. Whelan, J. I. Gorman, M. L. Whitelaw, Science 2002, 295, 858.

^{viii}B. Gu, C. Liu, J. Lin-Goerke, D. R. Maley, L. L. Gutshall, C. A. Feltenberger, A. M. del Vecchio, J. Viral. 2000, 74, 1794.

1. A process for the preparation of a compound of the formula I: 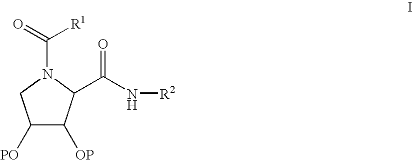
comprising reacting a compound of the formula II: 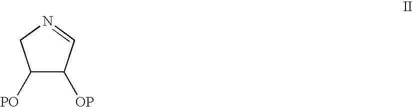
with a compound of the formula: 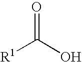
and a compound of the formula: 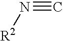
where R¹ is substituted or unsubstituted alkyl, alkenyl, or alkynyl, or an aromatic or non-aromatic cyclic or heterocyclic structure,
R² is substituted or unsubstituted alkyl or cycloalkyl, and each P is independently a protecting group.

2. A process according to claim 1, where R¹ is substituted or unsubstituted C1-C8 alkyl, alkenyl or alkynyl.

3. A process according to claim 1, wherein R¹ is substituted or unsubstituted C1-C6 alkyl, alkenyl or alkynyl.

4. A process according to claim 1, wherein R¹ is substituted or unsubstituted C1-C4 alkyl, alkenyl or alkynyl.

5. A process according to claim 1, wherein R¹ is an alkyl group substituted by a group of the formula:
R³ CO NH CH R⁴—
where R³ and R⁴ are each independently C1-C4 alkyl, phenyl or benzyl.

6. A process according to claim 1 where R¹ includes a substituted or unsubstituted 5- or 6-membered ring structure.

7. A process according to claim 6, wherein the ring is an alicyclic ring or includes at least one oxygen or nitrogen atom.

8. A process according to claim 1, wherein R¹ is n-propyl, phenyl or is a group of the formula:

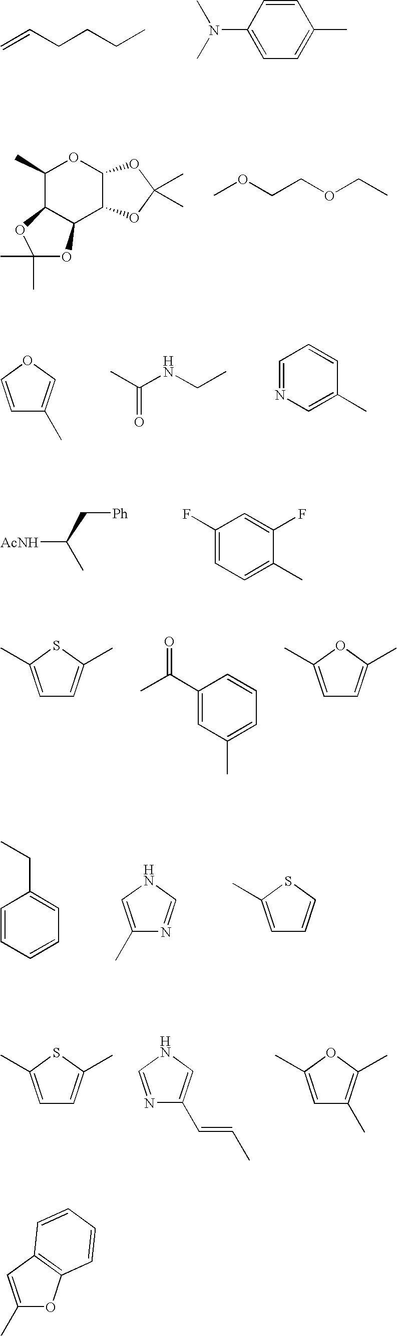

9. A process according to claim 1, wherein R² is substituted or unsubstituted C1-C10 alkyl.

10. A process according to claim 9, wherein R² is C1-C10 alkyl substituted by phenyl.

11. A process according to claim 1, wherein R² is i-propyl, n-butyl, t-butyl, n-pentyl, benzyl, cyclohexyl or a group of the formula:

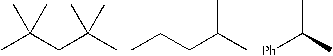

12. A process according to claim 1, wherein P is i-propyl or TBDMS.

13. A process according to claim 1, wherein the reaction is carried out in the presence of a non-aqueous solvent.

14. A process according to claim 13, wherein the solvent is methanol.

15. A process according to claim 1, wherein the compound of formula I is prepared by dehydrohalogenation of a compound of the formula: 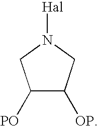
where Hal is halogen.

16. A process according to claim 15, where Hal is chlorine.

17. A process according to claim 15, wherein the dehydrohalogenation is carried out in the presence of DBU.

18. A process according to claim 17, wherein the dehydrohalogenation is carried out in the presence of a non-aqueous solvent.

19. A process according to claim 18, wherein the non-aqueous solvent is THF.

20. A process according to claim 1, wherein the compound of formula I is treated to remove the groups P.

21. A process according to claim 20, wherein the treatment is with acid.

22. A process according to claim 21, wherein the acid is trifluoroacetic acid.

23. A compound of the formula I 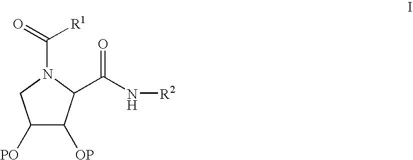
or of the formula II: 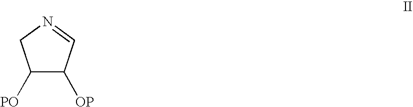
where R¹ is substituted or unsubstituted alkyl, alkenyl, or alkynyl, or an aromatic or non-aromatic cyclic or heterocyclic structure,
R² is substituted or unsubstituted alkyl or cycloalkyl, and each P is independently a protecting group.

24. A compound of the formula III: 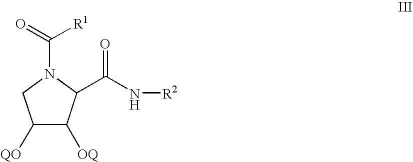
where R¹ and R² are as defined in claim 1; and
each Q is independently selected from hydrogen, a salt, protecting group or pharmaceutically acceptable prodrug thereof.

25. A compound according to claim 24, wherein R¹ is substituted or unsubstituted C1-C8 alkyl, alkenyl or alkynyl.

26. A compound according to claim 24, wherein R¹ is substituted or unsubstituted C1-C6 alkyl, alkenyl or alkynyl.

27. A compound according to claim 24, wherein R¹ is substituted or unsubstituted C1-C4 alkyl, alkenyl or alkynyl.

28. A compound according to claim 24, wherein R¹ is an alkyl group substituted by a group of the formula:
R³ CO NH CH R⁴—
wherein R³ and R⁴ are each independently C1-C4 alkyl, phenyl or benzyl.

29. A compound according to claim 24, wherein R¹ includes a substituted or unsubstituted 5- or 6-membered ring structure.

30. A compound according to claim 29, wherein the ring is an alicyclic ring or includes at least one oxygen or nitrogen atom.

31. A compound according to claim 24, wherein R¹ is n-propyl, phenyl or is a group of the formula:

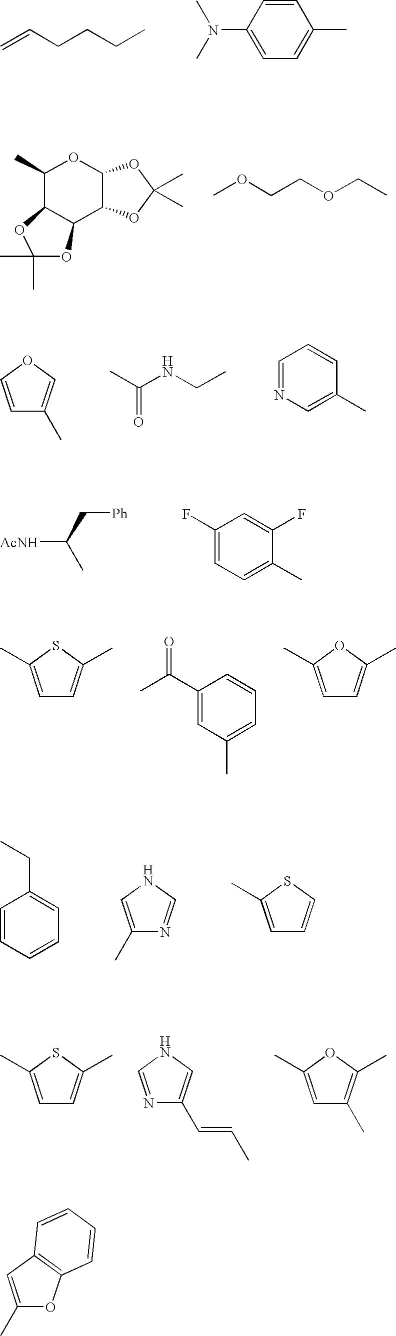

32. A compound according to claim 24, wherein R² is substituted or unsubstituted C1-C10 alkyl.

33. A compound according to claim 32, wherein R² is C1-C10 alkyl substituted by phenyl.

34. A compound according to claim 24, wherein R² is i-propyl, n-butyl, t-butyl, n-pentyl, benzyl, cyclohexyl or a group of the formula:

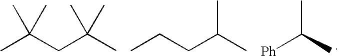

35. A chemical library comprising two or more different compounds of the formula III of claim 24.

36. A method of identifying a member of the library of claim 35 as an active agent against a particular target, including bringing the library into contact with said target and then determining the effect of each member of the library against a selected property of the target.

37. A method according to claim 36, wherein the target is a sugar- or peptide-based target.

38. A method according to claim 37, wherein the target is a glycosidase or is glycosyltransferase.

39. A method according to claim 38, wherein the glycosyltransferase is glucosylceramide synthase.

40. A method according to claim 37, wherein the target is an HIF hydroxylase or an elastase.

41. A method according to claim 37, wherein the target is hepatitis B virus, hepatitis C virus or bovine diarrhoea virus.

42. A compound as claimed in claim 24 for use as a medicament.

43. A pharmaceutical composition comprising a compound as claimed in claim 24 in combination with a pharmaceutically acceptable carrier, diluent or excipient.

44. Use of a compound as claimed in claim 24 in the manufacture of a medicament for the treatment of a disease with which a target of the compound is associated.

45. Use as claimed in claim 44, wherein the target is one associated with carbohydrate processing or peptide processing.

46. Use of a compound as claimed in claim 24 in the manufacture of a medicament for the treatment of a lipid storage disease or cancer.

47. Use as claimed in claim 46, wherein the lipid storage disease is Gaucher's disease.

48. Use of a compound as claimed in claim 24 in the manufacture of a medicament for the treatment of a viral infection.

49. Use as claimed in claim 48, wherein the viral infection is caused by a virus selected from the group consisting of hepatitis B, hepatitis C and bovine diarrhoea virus.
